(19) United States
(12) Patent Application Publication
(10) Pub. No.: US 2022/0305111 A1
(43) Pub. Date: Sep. 29, 2022

(54) IMMUNOBIOLOGICAL AGENT FOR INDUCING SPECIFIC IMMUNITY AGAINST SEVERE ACUTE RESPIRATORY SYNDROME VIRUS SARS-COV-2

(71) Applicant: FEDERAL STATE BUDGETARY INSTITUTION "NATIONAL RESEARCH CENTER FOR EPIDEMIOLOGY AND MICROBIOLOGY, Moscow (RU)

(72) Inventors: Olga Vadimovna ZUBKOVA, Khimki (RU); Tatiana Andreevna OZHAROVSKAIA, Korolev (RU); Inna Vadimovna DOLZHIKOVA, Lobnya (RU); Olga POPOVA, Ufa (RU); Dmitrii Viktorovich SHCHEBLIAKOV, Moscow (RU); Daria Mikhailovna GROUSOVA, Moscow (RU); Alina Shahmirovna DZHARULLAEVA, Moscow (RU); Amir Ildarovich TUKHVATULIN, Moscow (RU); Natalia Mikhailovna TUKHVATULINA, Moscow (RU); Dmitrii Nikolaevich SHCHERBININ, Nakhabino (RU); Ilias Bulatovich ESMAGAMBETOV, Dmitrov (RU); Elizaveta Alexandrovna TOKARSKAYA, Moscow (RU); Andrei Gennadevich BOTIKOV, Moscow (RU); Sergey Vladimirovich BORISEVICH, Sergiyev Posad (RU); Boris Savelievich NARODITSKY, Moscow (RU); Denis Yuryevich LOGUNOV, Moscow (RU); Aleksandr Leonidovich GINTSBURG, Moscow (RU)

(73) Assignee: FEDERAL STATE BUDGETARY INSTITUTION "NATIONAL RESEARCH CENTER FOR EPIDEMIOLOGY AND MICROBIOLOGY, Moscow (RU)

(21) Appl. No.: 17/427,745

(22) PCT Filed: Jul. 13, 2020
(86) PCT No.: PCT/RU2020/000344

§ 371 Date: Aug. 2, 2021

(30) Foreign Application Priority Data
Apr. 23, 2020 (RU) 2020114424

(51) Int. Cl. A61K 39/215; C12N 7/00; A61P 31/14

(52) U.S. Cl. CPC A61K 39/215; C12N 7/00; A61P 31/14

(57) ABSTRACT

The invention relates to biotechnology, immunology and virology and, in particular, to an immunobiological agent for the prevention of diseases caused by severe acute respiratory syndrome virus SARS-CoV-2. Also, a method of inducing specific immunity to the SARS-CoV-2 virus is disclosed, comprising the administration to mammals of one or more immunobiological agents for the prevention of diseases caused by severe acute respiratory syndrome virus SARS-CoV-2. The invention facilitates an effective induction of the immune response to the SARS-CoV-2 virus.

An immunobiological agent and a method of its use for the induction of specific immunity against the severe acute respiratory syndrome virus SARS-CoV-2 (variants).

### FIELD OF TECHNOLOGY

The invention relates to biotechnology, immunology and virology. The proposed remedy can be used for the prevention of diseases caused by the virus of severe acute respiratory syndrome SARS-CoV-2.

### STATE OF THE ART

SARS-CoV-2 is a new strain of coronavirus isolated at the end of 2019 in Wuhan (China), which has spread around the world in a few months. In January 2020, the World Health Organization declared the SARS-CoV-2 epidemic an international health emergency, and in March 2020, it described the spread of the disease as a pandemic. At the beginning of April 2020, the number of cases exceeded 1 million people, and the number of deaths—60 thousand people.

The disease that causes SARS-CoV-2 has received its own name COVID-19. This is a potentially severe acute respiratory infection, which can occur in both mild and severe forms and be accompanied by complications such as pneumonia, acute respiratory distress syndrome, acute respiratory failure, acute heart failure, acute renal failure, septic shock, cardiomyopathy, etc.

SARS-CoV-2 is spread by human-to-human transmission by airborne droplets or by direct contact. The reproductive index of SARS-CoV-2 (Basic reproduction number, RO), i.e. the number of people who become infected from one infected person, according to various sources is from 2.68 (Wu J T, Leung K, Leung G M. Nowcasting and forecasting the potential domestic and international spread of the 2019-nCoV outbreak originating in Wuhan, China: a modelling study. Lancet. 2020) to 6.6 (Sanche S, Lin Y T, Xu C, Romero-Severson E, Hengartner N, Ke R. The Novel Coronavirus, 2019-nCoV, is Highly Contagious and More Infectious Than Initially Estimated. medRxiv. 2020), and the average incubation period is 5.2 days (Li Q, Guan X, Wu P, Wang X, Zhou L, Tong Y. et al. Early Transmission Dynamics in Wuhan, China, of Novel Coronavirus-Infected Pneumonia. N Engl J Med. 2020).

Phylogenetic studies of strains isolated from COVID-19 patients have shown that the viruses closest to SARS-CoV-2 are found in bats (Zhou P. et al. A pneumonia outbreak associated with a new coronavirus of probable bat origin. Nature. 2020; 579: 270-273). It is also suggested that other mammalian species may be “intermediate” hosts in which SARS-CoV-2 was able to acquire some or all of the mutations necessary for effective transmission to humans (Zhang Y Z, Holmes E C. A Genomic Perspective on the Origin and Emergence of SARS-CoV-2. Cell. 2020 Mar. 26.)

The high mortality rate, the rapid geographical spread of SARS-CoV-2 and the vaguely defined etiology of the disease have created an urgent need to create effective means of preventing and treating diseases caused by this virus.

Over the past years, many efforts have been made to create various vaccines against coronavirus infections. The developed candidate vaccines can be classified into six types: 1) vaccines based on viral vectors; 2) DNA vaccines; 3) subunit vaccines; 4) nanoparticle-based vaccines; 5) vaccines based on inactivated whole virus 6) live attenuated vaccines. These vaccines were based on various viral proteins, such as nucleocapsid protein N, envelope protein E, NSP16 protein, S coronavirus protein (Ch. Yong et al. Recent Advances in the Vaccine Development Against Middle East Respiratory Syndrome-Coronavirus. Front Microbiol. 2019 Aug. 2; 10:1781.). Some of these drugs are at the stage of clinical trials (https://www.clinicaltrials.gov/). However, these drugs are not effective against the new SARS-CoV-2 virus, this is mainly due to the low homology of this coronavirus with the pathogens of human diseases SARS-CoV and MERS-CoV. For example, the degree of homology between the S protein SARS-CoV-2 and SARS-CoV is only 76% (Xu X, Chen P, Wang J, Feng J, Zhou H, Li X, et al. Evolution of the novel coronavirus from the ongoing Wuhan outbreak and modeling of its spike protein for risk of human transmission. Sci China Life Sci. 2020; 63(3):457-60). Thus, at the moment there is no registered vaccine against diseases caused by SARS-COV-2.

A solution is known for the U.S. Pat. No. 7,452,542B2, which proposes the use of a live attenuated coronavirus vaccine, in which the specified virus is characterized as containing a genome encoding the EXON polypeptide, including a replacement for tyrosine6398 MHV-A59 or its similar position, and the Orf2a polypeptide containing a replacement for leucine106 MHV-A59 or its similar position, and a pharmaceutically acceptable diluent.

A solution is known under the patent CN100360557C, which describes the use of the S protein of the SARS virus, which has a mutation in one of the positions: 778D→Y; 77D→G; 244T→I; 1182K→Q; 360F→S; 479N→R or K; 480D→G; 609A→L for the production of a vaccine against severe acute respiratory syndrome. The priority date of the application is Oct. 7, 2003.

A solution is known for the application for the invention US20080267992A1, which describes a vaccine against severe acute respiratory syndrome based on a recombinant human adenovirus 5 serotype containing a sequence of the complete protective antigen S of the SARS-CoV virus, or a sequence that includes the 51 domain of the SARS-CoV virus antigen S or the S2 domain of the SARS-CoV virus antigen S, or both domains. In addition, this recombinant adenovirus in the expression cassette contains a human cytomegalovirus promoter (CMV promoter) and a bovine growth hormone polyadenylation signal (polyA BGH).

This patent as the closest in terms of technical solution was chosen by the authors of the claimed invention for the prototype. A significant disadvantage of this solution is the use of virus antigens of another type of the coronavirus family.

Thus, there is an urgent need in the state of the art to develop a new immunobiological agent that provides the induction of an effective immune response against the SARS-CoV-2 coronavirus.

### DISCLOSURE OF THE INVENTION

The purpose of the claimed group of inventions is to create an immunobiological agent for the effective induction of an immune response against the SARS-CoV-2 virus.

The technical result is to create an effective means for the induction of specific immunity to SARS-Cov-2.

The specified technical result is achieved by creating an immunobiological agent for the prevention of diseases caused by the SARS-CoV-2 severe respiratory syndrome virus based on recombinant human adenovirus of the 5th serotype or recombinant human adenovirus of the 26th serotype, containing a sequence of the protective antigen S of the SARS-CoV-2 virus optimized for expression in mammalian cells with a deletion of 18 amino acids at the C′-end of the gene (SEQ ID NO:2).

Also, this technical result is achieved by creating an immunobiological agent for the prevention of diseases caused by the SARS-CoV-2 severe respiratory syndrome virus based on recombinant human adenovirus of the 5th serotype or recombinant human adenovirus of the 26th serotype, containing a sequence of the receptor-binding domain of the S protein of the SARS-CoV-2 virus with a sequence of the virus leader peptide (SEQ ID NO:4) optimized for expression in mammalian cells.

Also, this technical result is achieved by creating an immunobiological agent for the prevention of diseases caused by the SARS-CoV-2 severe respiratory syndrome virus based on recombinant human adenovirus of the 5th serotype or recombinant human adenovirus of the 26th serotype, containing a sequence of the receptor-binding domain of the S protein of the SARS-CoV-2 virus with the transmembrane domain of the vesicular stomatitis virus glycoprotein optimized for expression in mammalian cells (SEQ ID NO:5).

Also, this technical result is achieved by creating an immunobiological agent for the prevention of diseases caused by the SARS-CoV-2 severe respiratory syndrome virus based on recombinant human adenovirus of the 5th serotype or recombinant human adenovirus of the 26th serotype, containing a sequence of the receptor-binding domain of the S protein of the SARS-CoV-2 virus with a sequence of a leader peptide and a sequence of an Fc fragment from human IgG1 optimized for expression in mammalian cells (SEQ ID NO:6).

Also, this technical result is achieved by creating an immunobiological agent for the prevention of diseases caused by the SARS-CoV-2 severe respiratory syndrome virus based on recombinant human adenovirus of the 5th serotype or recombinant human adenovirus of the 26th serotype, containing a sequence of the complete protective antigen S of the SARS-CoV-2 virus optimized for expression in mammalian cells based on the gene sequences of the SARS-CoV-2 virus protein S (SEQ ID NO:1) in combination with immunobiological agents (SEQ ID NO: 2), and/or (SEQ ID NO: 3), and/or (SEQ ID NO: 4), and/or (SEQ ID NO:5), and/or (SEQ ID NO: 6).

Also, the specified technical result is achieved by the method of induction of specific immunity to the SARS-CoV-2 virus, including the introduction into the mammalian body of one or more agents (SEQ ID NO: 1), and/or (SEQ ID NO: 2), and/or (SEQ ID NO: 3), and/or (SEQ ID NO: 4), and/or (SEQ ID NO:5), and/or (SEQ ID NO:6) in an effective amount.

Also, this technical result is achieved by inducing specific immunity to the SARS-CoV-2 virus, where two different immunobiological agents based on recombinant human adenovirus of the 5th serotype or two different immunobiological agents based on recombinant human adenovirus of the 26th serotype are sequentially injected into the mammalian body with an interval of more than 1 week.

Also, this technical result is achieved by inducing specific immunity to the SARS-CoV-2 virus, where any of the immunobiological agents based on recombinant human adenovirus of serotype 5 and any of the immunobiological agents based on recombinant human adenovirus of the serotype 26 are sequentially injected into the mammalian body with an interval of more than 1 week, or in the sequential introduction into the mammalian body of any of the immunobiological agents based on the recombinant human adenovirus of serotype 26 and any of the immunobiological agents based on the recombinant human adenovirus of the serotype 5 at intervals of more than 1 week.

Also, this technical result is achieved by inducing specific immunity to the SARS-CoV-2 virus, where any two immunobiological agents based on recombinant human adenovirus of the serotype 5 or 26 are simultaneously injected into the mammalian body.

The essence of the claimed group of inventions is explained by the drawings, where FIGS. 1-5 show the results of evaluating the effectiveness of immunization.

### IMPLEMENTATION OF THE INVENTION

### Brief Description of the Figures

FIG. 1 the results of evaluation of the effectiveness of immunization developed by immunological means on the basis of recombinant adenovirus containing optimized for expression in mammalian cells the sequence of the protective antigen (protein S, RBD, S-del, S-Fc, RBD-G, RBD-Fc) SARS-CoV-2 with a sequence selected from SEQ ID NO:1, SEQ ID NO:2, SEQ ID NO:3, SEQ ID NO:4, SEQ ID NO:5, SEQ ID NO:6 according to the estimation of the proportion of proliferating CD4+ lymphocytes restimulated by SARS-CoV-2 glycoprotein S on day 8 after immunization of the test animals.

Ordinate axis—the number of proliferating cells, %

The abscissa axis—various groups of animals:
1) phosphate buffer (100 μl)
2) Ad5-S-CoV-2 108BOE/mouse
3) Ad5-S-del-CoV-2 108BOE/mouse
4) Ad5-S-Fc-CoV-2 108BOE/mouse
5) Ad5-RBD-CoV-2 108BOE/mouse
6) Ad5-RBD-G-CoV-2 108BOE/mouse
7) Ad5-RBD-Fc-CoV-2 108BOE/mouse

FIG. 2 the results of evaluation of the effectiveness of immunization developed by immunological means on the basis of recombinant adenovirus containing optimized for expression in mammalian cells the sequence of the protective antigen (protein S, RBD, S-del, S-Fc, RBD-G, RBD-Fc) SARS-CoV-2 with a sequence selected from SEQ ID NO:1, SEQ ID NO:2, SEQ ID NO:3, SEQ ID NO:4, SEQ ID NO:5, SEQ ID NO:6 according to the estimation of the proportion of proliferating CD4+ lymphocytes restimulated by SARS-CoV-2 glycoprotein S on day 15 after immunization of the test animals.

Ordinate axis—the number of proliferating cells, %

The abscissa axis—various groups of animals:

1) phosphate buffer (100 μl)

2) Ad5-S-CoV-2 108BOE/mouse

3) Ad5-S-del-CoV-2 108BOE/mouse

4) Ad5-S-Fc-CoV-2 108BOE/mouse

5) Ad5-RBD-CoV-2 108BOE/mouse

6) Ad5-RBD-G-CoV-2 108BOE/mouse

7) Ad5-RBD-Fc-CoV-2 108BOE/mouse

FIG. 3 The results of evaluation of the effectiveness of immunization developed by immunological means on the basis of recombinant adenovirus containing optimized for expression in mammalian cells the sequence of the protective antigen (protein S, RBD, S-del, S-Fc, RBD-G, RBD-Fc) SARS-CoV-2 with a sequence selected from SEQ ID NO:1, SEQ ID NO:2, SEQ ID NO:3, SEQ ID NO:4, SEQ ID NO:5, SEQ ID NO:6 according to the estimation of the proportion of proliferating CD8+ lymphocytes restimulated by SARS-CoV-2 glycoprotein S on day 8 after immunization of the test animals.

Ordinate axis—the number of proliferating cells, %

The abscissa axis—various groups of animals:

1) phosphate buffer (100 μl)

2) Ad5-S-CoV-2 108BOE/mouse

3) Ad5-S-del-CoV-2 108BOE/mouse

4) Ad5-S-Fc-CoV-2 108BOE/mouse

5) Ad5-RBD-CoV-2 108BOE/mouse

6) Ad5-RBD-G-CoV-2 108BOE/mouse

7) Ad5-RBD-Fc-CoV-2 108BOE/mouse

FIG. 4 the results of evaluation of the effectiveness of immunization developed by immunological means on the basis of recombinant adenovirus containing optimized for expression in mammalian cells the sequence of the protective antigen (protein S, RBD, S-del, S-Fc, RBD-G, RBD-Fc) SARS-CoV-2 with a sequence selected from SEQ ID NO:1, SEQ ID NO:2, SEQ ID NO:3, SEQ ID NO:4, SEQ ID NO:5, SEQ ID NO:6 according to the estimation of the proportion of proliferating CD8+ lymphocytes restimulated by SARS-CoV-2 virus glycoprotein S on the 15th day after immunization of the test animals.

Ordinate axis—the number of proliferating cells, %

The abscissa axis—various groups of animals:

1) phosphate buffer (100 μl)

2) Ad5-S-CoV-2 108BOE/mouse

3) Ad5-S-del-CoV-2 108BOE/mouse

4) Ad5-S-Fc-CoV-2 108BOE/mouse

5) Ad5-RBD-CoV-2 108BOE/mouse

6) Ad5-RBD-G-CoV-2 108BOE/mouse

7) Ad5-RBD-Fc-CoV-2 108BOE/mouse

In FIG. 5 the results of evaluating the effectiveness of the developed immunobiological agent based on a recombinant adenovirus containing a sequence of protective antigen (proteins S, RBD, S-del, S-Fc, RBD-G, RBD-Fc) SARS-CoV-2 optimized for expression in mammalian cells with a sequence selected from SEQ ID NO: 1, SEQ ID NO: 2, SEQ ID NO: 3, SEQ ID NO: 4, SEQ ID NO: 5, SEQ ID NO:6 according to the assessment of the increase in the concentration of IFN-gamma in the medium after stimulation of splenocytes of C57/BL6 mice immunized with adenovirus constructs with a recombinant full-size S protein of the SARS-CoV-2 virus, on day 15 after immunization of the test animals.

The ordinate axis is the values of the increase in the concentration of IFN-gamma in the medium of stimulated cells when compared with intact cells (times).

The abscissa axis—the studied groups of animals: intact animals and animals that were injected with 108BOE/mouse

1) phosphate buffer (100 μl)

2) Ad5-S-CoV-2 108BOE/mouse

3) Ad5-S-del-CoV-2 108BOE/mouse

4) Ad5-S-Fc-CoV-2 108BOE/mouse

5) Ad5-RBD-CoV-2 108BOE/mouse

6) Ad5-RBD-G-CoV-2 108BOE/mouse

7) Ad5-RBD-Fc-CoV-2 108BOE/mouse

The first stage in the development of an immunobiological agent against the SARS-CoV-2 coronavirus was the choice of a vaccine antigen. In the course of the work, a literary search was conducted, which showed that the most promising antigen for creating a candidate vaccine is the S protein of the coronavirus. This is a type I transmembrane glycoprotein, which is responsible for the binding, fusion and penetration of viral particles into the cell. It has been shown to be an inducer of neutralizing antibodies (Liang M et al, SARS patients-derived human recombinant antibodies to S and M proteins effectively neutralize SARS-coronavirus infection. Biomed Environ Sci. 2005 December; 18(6):363-74).

The S protein consists of a signal peptide (amino acids 1-12) and 3 domains: an extracellular domain (amino acids 13-1193), a transmembrane domain (amino acids 1194-1215), an intracellular domain (amino acids 1216-1255). The extracellular domain consists of 2 subunits 51 and S2, and a small area between them, the functions of which are not completely clear. The 51 subunit is responsible for binding the virus to the ACE2 receptor (angiotensin-converting enzyme 2). The site that is located in the middle region of the S1 subunit (amino acids 318-510) is called the receptor-binding domain (RBD). The S2 subunit, which contains a putative fusion peptide and two heptad repeats (HR1 and HR2), is responsible for the fusion between the virus and the target cell membrane. The infection is initiated by the binding of the RBD subunit S1 of the virus to the cellular receptor ACE2. After that, a fusion core is formed between the HR1 and HR2 regions of the S2 subunit, which entails the convergence of the viral and cell membranes, which as a result merge and the virus enters the cell. Therefore, the use of S protein or its fragments in the composition of the vaccine can induce antibodies that block the penetration of the virus into the cell.

To achieve the most effective induction of immune reactions, the authors proposed various variants of modifications of this antigen, as well as the possibility of its combination with the transmembrane domain of the vesicular stomatitis virus glycoprotein to increase the level of expression of the target protein.

6 different variants of nucleotide sequences (of the modified S gene of the SARS-CoV-2 virus or the receptor-binding domain of the S protein) were obtained by optimizing these sequences for expression in mammalian cells.

Further, several constructs based on recombinant human adenoviruses of serotypes 5 and 26 were developed for the effective delivery of modified genes to mammalian cells. Adenoviral vectors were chosen because they have such advantages as safety, a wide range of tissue tropism, a well-characterized genome, ease of genetic manipulation, the ability to include large inserts of transgenic DNA, their inherent adjuvant properties, the ability to induce a stable T-cell and humoral response.

Of the number of known adenoviruses, the most studied are human adenoviruses of serotype 5, so they became the basis for creating vectors for gene therapy. Technologies have been developed for obtaining vectors of the first and second generations, chimeric vectors (containing proteins of viruses of other serotypes) (J. N. Glasgow et al., The vector of adenovirus with a chimeric fiber obtained from canine adenovirus type 2, demonstrates a new tropism, Virology, 2004, No. 324, 103-116) and a number of other vectors. Also, vectors derived from other serotypes were created, for example, the 26th (X). Chen et al. al., Adenovirus-based vaccines: comparison of vectors from three types of adenoviruses, Virology, 2010, No 84(20), 10522-10532).

Vectors based on human adenovirus of the serotype 26 show a high level of immunogenicity in primates, where they are able to induce a powerful CD8+ T-cell response that qualitatively exceeds the T-cell response when vectors based on human adenovirus of the 5th serotype are introduced into the body (J. Liu et. al., Magnitude and phenotype of cellular immune responses elicited by recombinant adenovirus vectors and heterologous prime-boost regimens in rhesus monkeys, Virology, 2008, No. 82, 4844-4852). At the same time, a larger number of epitopes are recognized and the production of a wider range of factors is induced, rather than mainly interferon gamma (J. Liu et. al., Magnitude and phenotype of cellular immune responses elicited by recombinant adenovirus vectors and heterologous prime-boost regimens in rhesus monkeys, Virology, 2008, No. 82, 4844-4852). These data suggest that vectors based on human adenovirus of the 26th serotype have fundamental differences in the ability to induce the formation of an immune response to the target antigen relative to other adenovirus vectors.

The invention according to variant 2 is a recombinant human adenovirus of the 5th serotype, or a recombinant human adenovirus of the 26th serotype, containing a sequence of the complete protective antigen S SARS-CoV-2 optimized for expression in mammalian cells with a deletion of 18 amino acids at the C′ end of the gene (SEQ ID NO:2).

The invention according to variant 2 is a recombinant human adenovirus of the serotype 5, or a recombinant human adenovirus of the serotype 26, containing a sequence of the complete protective antigen S SARS-CoV-2 optimized for expression in mammalian cells with a deletion of 18 amino acids at the C′ end of the gene (SEQ ID NO:2).

A method for inducing specific immunity to the SARS-CoV-2 virus has been developed, including the introduction of one or more drugs into the mammalian body according to variants 1-6 in an effective amount. This method provides for:

1) sequential introduction into the mammalian body of two different immunobiological agents based on recombinant human adenovirus of serotype 5 or two different immunobiological agents based on recombinant human adenovirus of the serotype 26 according to variants 1-6 with an interval of more than 1 week

2) sequential introduction into the mammalian body of any of the immunobiological agents based on recombinant human adenovirus of the 5th serotype and any of the immunobiological agents based on recombinant human adenovirus of the 26th serotype according to variants 1-6 with an interval of more than 1 week, or in the sequential introduction into the mammalian body of any of the immunobiological agents based on the recombinant human adenovirus of the serotype 26 and any of the immunobiological agents based on the recombinant human adenovirus of the serotype 5 according to variants 1-6 with an interval of more than 1 week.

3) Simultaneous introduction into the mammalian body of any two immunobiological agents based on recombinant human adenovirus of the serotype 5 or 26 according to claim 1, and/or claim 2, and/or claim 3, and/or claim 4, and/or claim 5, and/or claim 6.

The implementation of the invention is confirmed by the following examples.

### EXAMPLE 1. PREPARATION OF VARIOUS VARIANTS OF SARS-COV-2 GLYCOPROTEIN S

At the first stage of the work, the authors developed several modifications of the vaccine antigen to achieve the most effective immune response.

The S protein of the SARS-CoV-2 virus with the sequence SEQ ID NO:1 was taken as a basis, which was then modified in several ways:

1) To represent protein S on the plasma membrane, 18 amino acids were deleted at the C′-end of protein S (S-del) SEQ ID NO:2 (used for variant 2).

2) In addition, the sequence of the complete protective antigen S of the SARS-CoV-2 virus with the sequence of the Fc fragment from human IgG1 was optimized for expression in mammalian cells (used for option 3). This modification enhances immunogenicity due to the possible binding of the Fc protein fragment to the Fc receptor on antigen-presenting cells (Li Z., Palaniyandi S., Zeng R., Tuo W., Roopenian D. C., Zhu X., Transfer of IgG in the female genital tract by MHC class I-related neonatal Fc receptor (FcRn) confers protective immunity to vaginal infection. Proc. Natl. Acad. Sci. U.S.A, 2011, No. 108, 4388-93), and also increases the stability of the protein and prolongs its half-life in vivo (Zhang M. Y., Wang Y., Mankowski M. K., Ptak R. G., Dimitrov D. S., Cross-reactive HIV-1-neutralizing activity of serum IgG from a rabbit immunized with gp41 fused to IgG1 Fc: Possible role of the prolonged half-life of the immunogen, Vaccine, 2009, No. 27, 857-863).

3) To study the immunogenicity of only the receptor-binding domain (RBD) of the SARS-CoV-2 virus protein in the secreted form, a sequence SEQ ID NO:4 was created (used for variant 4) containing a sequence of the receptor-binding domain of protein S with a sequence of a leader peptide (added for protein secretion).

4) To study the RBD protein S of the SARS-CoV-2 virus in an unclassified form, the sequence SEQ ID NO was selected: 5 (used for variant 5), consisting of the RBD protein S of the SARS-CoV-2 virus, to which the sequence of the transmembrane domain of the vesicular stomatitis virus glycoprotein (RBD-G) was added.

5) To study the secreted form of RBD protein S with the sequence of the leader peptide and the sequence of the Fc fragment from human IgG1, the sequence SEQ ID NO was selected: 6 (used for option 6). The addition of an Fc fragment from human IgG1 enhances immunogenicity due to the possible binding of the Fc fragment of the protein to the Fc receptor on antigen-presenting cells (Z. Li et. al., Transfer of IgG in the female genital tract by MHC class I-related neonatal Fc receptor (FcRn) confers protective immunity to vaginal infection, Proceedings of the National Academy of Sciences USA, 2011, No. 108, 4388-4393), and can also enhance protein stability and prolong the half-life in vivo (M. Y. Zhang et. al., Crossreactive HIV-1-neutralizing activity of serum IgG from a rabbit immunized with gp41 fused to IgG1 Fc: Possible role of the prolonged half-life of the immunogen, Vaccine, 2008, No. 27, 857-63).

### EXAMPLE 2. OBTAINING GENETIC CONSTRUCTS ENCODING THE S PROTEIN GENE IN VARIOUS VARIANTS

At the next stage of the work, the amino acid sequences according to example 1 (SEQ ID NO: 1, SEQ ID NO: 2, SEQ ID NO: 3, SEQ ID NO: 4, SEQ ID NO:5, SEQ ID NO:6) were translated into nucleotide sequences. Further, the obtained sequences were optimized for expression in mammalian cells. All the nucleotide sequences were obtained by the synthesis method of CJSC “Eurogen” (Moscow). As a result, the following genetic constructs were obtained:

1) pVax-S-CoV-2 containing the nucleotide sequence of the complete S gene of the SARS-CoV-2 virus;

2) pVax-S-del-CoV-2 containing the nucleotide sequence of the S gene of the SARS-CoV-2 c virus with a deletion of 18 amino acids at the C′ end of the gene;

3) pVax-S-Fc-CoV-2, containing the nucleotide sequence of the complete S gene of the SARS-CoV-2 virus and the sequence of the Fc fragment from human IgG1

4) pAL2-T-RBD-CoV-2, containing the nucleotide sequence of the receptor-binding domain of the protein S with the sequence of the gene of the leader peptide;

5) pAL2-T-RBD-G-CoV-2 containing the nucleotide sequence of the receptor-binding domain of the protein S with the G gene of the vesicular stomatitis virus;

6) pAL2-T-RBD-Fc-CoV-2, containing the nucleotide sequence of the receptor-binding domain of the protein S with the sequence of the leader peptide gene and the nucleotide sequence of the Fc fragment from human IgG1.

Then, using genetic engineering methods, the S protein gene sequence from the pVax-S-CoV-2 construct was cloned using the XbaI restriction endonuclease into the shuttle plasmid pShuttle-CMV (StrataGen, USA) and the resulting plasmid was named pShuttle-S-CoV-2. Thus, the shuttle plasmid pShuttle-S-CoV-2 was created, carrying the nucleotide sequence of the amino acid sequence S (SEQ ID NO) optimized for expression in mammalian cells: 1), obtained in Example 1.

Similarly, the nucleotide sequences of modified variants of the S SARS-CoV-2 protein were cloned into the shuttle plasmid pShuttle-CMV (StrataGen, USA) and the following shuttle plasmids were obtained:
pShuttle-S-del-CoV-2 (contains an optimized nucleotide sequence of the S gene of the SARS-CoV-2 virus with a deletion of 18 amino acids at the C′-end);
pShuttle-S-Fc-CoV-2 containing the optimized nucleotide sequence of the complete S gene of the SARS-CoV-2 virus and the sequence of the Fc fragment from human IgG1;
pShuttle-RBD-CoV-2 (contains an optimized nucleotide sequence of the S SARS-CoV-2 receptor-binding domain);
pShuttle-RBD-G-CoV-2 (contains an optimized nucleotide sequence of the S SARS-CoV-2 receptor-binding domain with the transmembrane domain of the vesicular stomatitis virus glycoprotein);
pShuttle-RBD-Fc-CoV-2 (contains an optimized nucleotide sequence of the S SARS-CoV-2 receptor-binding domain with an optimized sequence of the Fc fragment from human IgG1).

### EXAMPLE 3. PREPARATION OF AN IMMUNOBIOLOGICAL AGENT BASED ON A RECOMBINANT HUMAN ADENOVIRUS OF THE SEROTYPE 5

At the next stage of the work, a recombinant adenovirus plasmid pAd5-S-CoV-2 was obtained, containing a sequence of the complete protective antigen S SARS-CoV-2 optimized for expression in mammalian cells (SEQ ID NO:1) (option 1). This plasmid was obtained by homologous recombination between the pAd-Easy plasmid (AdEasy™ Adenoviral Vector System, StratoGen, USA), containing the genomic part of human adenovirus 5 serotype with removed E1 and E3 regions, and the shuttle plasmid pShuttle-S (obtained in Example 3), carrying homologous sections of the adenovirus genome and an expression cassette with the target gene (protein S). To do this, the shuttle plasmid pShuttle-S obtained in Example 3 was linearized with the restriction endonuclease PmeI.

Homologous recombination was performed in E. coli cells of the BJ5183 strain. The AdEasy plasmid was mixed with the pShuttle-S plasmid, and then the resulting mixture was transformed into E. coli cells by electroporation according to the manual “MicroPulser™ Electroporation Apparatus Operating Instructions and Applications Guide” (Bio-Rad, USA). After transformation, the E. coli cells of the BJ5183 strain were sown on cups with LB-agar containing a selective antibiotic and grown for 18 hours at a temperature of +37° C. The transformation efficiency was 1010-1011 transformed clones per 1 microgram of the pBluescript II SK (−) plasmid.

As a result of homologous recombination, a cassette with the target transgen (protein S) appeared in the pAd-Easy plasmid, and the antibiotic resistance gene changed.

Thus, a recombinant adenovirus plasmid pAd5-S-CoV-2 was constructed, containing a full-size genome of a recombinant human adenovirus of the 5th serotype (with deleted E1 and E3 regions of the genome) with an integrated genetic construct obtained in Example 3. Next, the pAd5-S-CoV-2 plasmid was hydrolyzed with a restriction endonuclease Pac I and transfected a permissive cell culture with it human embryonic kidney of the NEK 293 line. The cells of the NEK 293 line contain in their genome an embedded E1 region of the human adenovirus genome of the 5th serotype, due to which recombinant replication-defective human adenoviruses of the 5th serotype can multiply in them. On the sixth day after transfection, the first blind passages were performed to obtain recombinant adenovirus more efficiently. After the onset of the cytopathic effect of the virus (microscopy data), the cells with the culture medium were frozen three times to destroy the cells and release the virus. As a result, a material was obtained, which was then used to accumulate preparative amounts of recombinant adenoviruses.

The activity of the pAd5-S-CoV-2 preparation was evaluated here and further by the standard titration method on a culture of sensitive 293 HEK cells in plaque formation reactions.

To confirm the design of the proposed recombinant pseudoadenovirus particle based on human adenovirus serotype 5 expressing the S gene of the SARS-CoV-2 virus, a polymerase chain reaction (PCR) was performed according to a well-known standard technique.

Similarly, five more recombinant adenoviruses were obtained: Ad5-S-del-CoV-2, Ad5-S-Fc-CoV-2, Ad5-RBD-CoV-2, Ad5-RBD-G-CoV-2, Ad5-RBD-Fc-CoV-2.

Thus, as a result of the work carried out, variants of an immunobiological agent based on a recombinant human adenovirus of the 5th serotype containing:

1) optimized nucleotide sequence of the S SARS-CoV-2 receptor-binding domain (variant 1);

2) optimized nucleotide sequence of the protective S antigen of the SARS-CoV-2 virus with a deletion of 18 amino acids at the C′-end of the gene (variant 2);

3) the sequence of the complete protective antigen S of the SARS-CoV-2 virus and the sequence of the Fc fragment from human IgG1 optimized for expression in mammalian cells (variant 3);

4) optimized nucleotide sequence of the receptor-binding domain of the protein S with the sequence of the leader peptide (variant 4),

5) optimized nucleotide sequence of the receptor-binding domain of protein S with the transmembrane domain of the vesicular stomatitis virus glycoprotein (variant 5),

6) an optimized sequence of the receptor-binding domain of the S protein with the sequence of the leader peptide and the sequence of the Fc fragment from the human IgG1 (variant 6).

### EXAMPLE 4. PREPARATION OF AN IMMUNOBIOLOGICAL AGENT BASED ON RECOMBINANT HUMAN ADENOVIRUS OF SEROTYPE 26

At the first stage, an expression cassette with the S SARS-CoV-2 gene was placed in the recombinant vector pAd26-ORF6-Ad5. To do this, the pAd26-ORF6-Ad5 vector was linearized using the PmeI restriction endonuclease, and the pShuttle-S plasmid construct obtained in Example 3 was processed with PmeI restriction endonucleases. The hydrolysis products were ligated, after which the pAd26-S-CoV-2 plasmid was obtained using standard methods.

At the next stage, the pAd26-S-CoV-2 plasmid was hydrolyzed with Paci and SwaI restriction endonucleases and transfected a permissive culture of NEK 293 cells with it. On the third day after transfection, the first blind passages were performed to obtain recombinant adenovirus more efficiently. After the onset of the cytopathic effect of the virus (microscopy data), the cells with the culture medium were frozen three times to destroy the cells and release the virus. As a result, a material was obtained, which was then used to accumulate preparative amounts of recombinant adenoviruses. The activity of the pAd26-S-CoV-2 preparation was evaluated here and further by the standard titration method on a 293 HEK cell culture in a plaque formation reaction.

To confirm the design of the proposed recombinant pseudoadenovirus particle based on recombinant human adenovirus of the 26th serotype expressing the SARS-CoV-2 gene, a polymerase chain reaction (PCR) was performed according to a well-known standard technique.

Similarly, five more recombinant adenoviruses were obtained: pAd26-S-dek-CoV-2, pAd26-S-Fc-CoV-2, pAd26-RBD-CoV-2, pAd26-RBD-G-CoV-2, pAd26-RBD-Fc-CoV-2.

Thus, as a result of the work carried out, variants of an immunobiological agent based on a recombinant human adenovirus of the 26th serotype containing:

1) optimized nucleotide sequence of the S SARS-CoV-2 receptor-binding domain (variant 1);

2) optimized nucleotide sequence of the protective S antigen of the SARS-CoV-2 virus with a deletion of 18 amino acids at the C′-end of the gene (variant 2);

3) the sequence of the complete protective antigen S of the SARS-CoV-2 virus and the sequence of the Fc fragment from human IgG1 optimized for expression in mammalian cells (variant 3);

4) optimized nucleotide sequence of the receptor-binding domain of the protein S with the sequence of the leader peptide (variant 4),

5) optimized nucleotide sequence of the receptor-binding domain of protein S with the transmembrane domain of the vesicular stomatitis virus glycoprotein (variant 5),

6) an optimized sequence of the receptor-binding domain of the S protein with the sequence of the leader peptide and the sequence of the Fc fragment from the human IgG1 (variant 6).

### EXAMPLE 5. CHECKING THE EXPRESSION OF VARIOUS VARIANTS OF THE SARS-COV-2 GLYCOPROTEIN S GENE IN HEK293 CELLS AFTER THE ADDITION OF AN IMMUNOBIOLOGICAL AGENT BASED ON RECOMBINANT HUMAN ADENOVIRUS OF SEROTYPE 5

The purpose of this experiment was to test the ability of the constructed recombinant adenoviruses Ad5-S-CoV-2, Ad5-S-del-CoV-2, Ad5-S-Fc-CoV-2, Ad5-RBD-CoV-2, Ad5-RBD-G-CoV-2, Ad5-RBD-Fc-CoV-2 to express various variants of the S protein gene in mammalian cells.

HEK293 cells were cultured in DMEM medium with the addition of 10% embryonic calf serum in an incubator at a temperature of 37° C. and 5% CO2. The cells were placed on 35 mm2 culture Petri dishes and incubated for a day until 70% confluence was achieved. Further, the studied preparations of recombinant adenoviruses (Ad5-S-CoV-2, Ad5-S-del-CoV-2, Ad5-S-Fc-CoV-2, Ad5-RBD-CoV-2, Ad5-RBD-G-CoV-2, Ad5-RBD-Fc-CoV-2) were added to the cells, a control drug (Ad5-null—recombinant adenovirus, which does not contain inserts) at the rate of 100 BOE/the cell and the phosphate-salt buffer (FSB) as a negative control. 2 days after the transduction, the cells were collected, lysed in 0.5 ml of a single CCLR buffer (Promega), the lysate was diluted with a carbonate-bicarbonate buffer and introduced into the wells of the ELISA tablet. The tablet was incubated during the night +4° C.

The wells of the tablet were washed with a single buffer for washing three times with a volume of 200 ml per well, and then 100 ml of blocking buffer were added, covered with a lid and incubated for 1 hour 37° C. on a shaker at 400 rpm. Next, the wells of the tablet were washed with a single buffer for washing three times with a volume of 200 μl per well and 100 μl of blood serum of convalescent was added. The tablet was covered with a lid and incubated at room temperature on a shaker at 400 rpm for 2 hours. Next, the wells of the tablet were washed with a single buffer for washing three times with a volume of 200 ml per well, then 100 ml of a solution of secondary antibodies conjugated with biotin was added. The tablet was covered with a lid and incubated at room temperature on a shaker at 400 rpm for 2 hours. Next, a solution of streptavidin conjugated with horseradish peroxidase was prepared. To do this, a conjugate with a volume of 60 μl was diluted in 5.94 ml of buffer for analysis. The wells of the tablet were washed twice with a single buffer for washing with a volume of 200 ml per well and 100 ml of streptavidin solution conjugated with horseradish peroxidase was added to all the wells of the tablet. The tablet was incubated at room temperature on a shaker at 400 revolutions per minute for 1 hour. Then the wells of the tablet were washed twice with a single buffer for washing with a volume of 200 μl per well and 100 μl of TMB of substrate was added to all the wells of the tablet and incubated in the dark at room temperature for 10 minutes, and then 100 μl of stopping solution was added to all the wells. The optical density value was determined by measurement on a flatbed spectrophotometer (Multiskan FC, Thermo) at a wavelength of 450 nm. The results of the experiment are presented in Table 1.

[symbol]

⁻ [symbol] [symbol] [symbol] [symbol] [symbol] [symbol] [symbol] [symbol] S SARS-COV-2 [symbol] [symbol] HEK293

[symbol]

[symbol]

[symbol]

[symbol]

[symbol]

[symbol]

[symbol]

[symbol]

[symbol]

[symbol]

[symbol]

| | The average value of the optical |
|---|---|
| | density at a wavelength of |
| | 450 nm |
| FSB | 0,19 (±0,05) |
| Ad5-null | 0,23 (±0,08) |
| Ad5-S-CoV-2 | 1,85 (±0,15) |
| Ad5-S-del-CoV-2, | 1,63 (±0,19) |
| Ad5-S-Fc-CoV-2 | 1,57 (±0,30) |
| Ad5-RBD-CoV-2 | 1,47 (±0,21) |
| Ad5-RBD-G-CoV-2 | 1,52 (±0,19) |
| Ad5-RBD-Fc-CoV-2 | 1,58± (0,11) |

[symbol]

[symbol]

[symbol]

[symbol]

[symbol]

[symbol]

[symbol]

[symbol]

[symbol]

[symbol]

Ad5-CoV-2, Ad5-S-del-CoV-2, Ad5-S-Fc-CoV-2, Ad5-
RBD-CoV-2, Ad5-RBD-G-CoV-2, Ad5-RBD-Fe-CoV-2

[symbol]

[symbol]

[symbol]

[symbol]

[symbol]

[symbol]

### EXAMPLE 6. CHECKING THE EXPRESSION OF VARIOUS VARIANTS OF THE SARS-COV-2 GLYCOPROTEIN S GENE IN HEK293 CELLS AFTER ADDING AN IMMUNOBIOLOGICAL AGENT BASED ON RECOMBINANT HUMAN ADENOVIRUS OF SEROTYPE 26

The purpose of this experiment was to test the ability of the constructed recombinant adenoviruses pAd26-S-CoV-2, Ad26-S-del-CoV-2, Ad26-S-Fc-CoV-2, pAd26-RBD-CoV-2, pAd26-RBD-G-CoV-2, pAd26-RBD-Fc-CoV-2 to express various variants of the S protein gene in mammalian cells.

HEK293 cells were cultured in DMEM medium with the addition of 10% embryonic calf serum in an incubator at a temperature of 37° C. and 5% CO2. The cells were placed on 35 mm2 culture Petri dishes and incubated for a day until 70% confluence was achieved. Further, the studied preparations of recombinant adenoviruses (pAd26-S-CoV-2, Ad26-S-del-CoV-2, Ad26-S-Fc-CoV-2, pAd26-RBD-CoV-2, pAd26-RBD-G-CoV-2, pAd26-RBD-Fc-CoV-2), a control preparation (Ad26-null—recombinant adenovirus that does not contain inserts) were added to the cells at the rate of 100 BOE/cell and phosphate-salt buffer (FSB) as a negative control. 2 days after the transduction, the cells were collected, lysed in 0.5 ml of a single CCLR buffer (Promega), the lysate was diluted with a carbonate-bicarbonate buffer and introduced into the wells of the ELISA tablet. The tablet was incubated during the night +4° C.

The wells of the tablet were washed with a single buffer for washing three times with a volume of 200 ml per well, and then 100 ml of blocking buffer were added, covered with a lid and incubated for 1 hour 37° C. on a shaker at 400 rpm. Next, the wells of the tablet were washed with a single buffer for washing three times with a volume of 200 μl per well and 100 μl of blood serum of convalescent was added. The tablet was covered with a lid and incubated at room temperature on a shaker at 400 rpm for 2 hours. Next, the wells of the tablet were washed with a single buffer for washing three times with a volume of 200 ml per well, then 100 ml of a solution of secondary antibodies conjugated with biotin was added. The tablet was covered with a lid and incubated at room temperature on a shaker at 400 rpm for 2 hours. Next, a solution of streptavidin conjugated with horseradish peroxidase was prepared. To do this, a conjugate with a volume of 60 μl was diluted in 5.94 ml of buffer for analysis. The wells of the tablet were washed twice with a single buffer for washing with a volume of 200 ml per well and 100 ml of streptavidin solution conjugated with horseradish peroxidase was added to all the wells of the tablet. The tablet was incubated at room temperature on a shaker at 400 revolutions per minute for 1 hour. Then the wells of the tablet were washed twice with a single buffer for washing with a volume of 200 μl per well and 100 μl of TMB of substrate was added to all the wells of the tablet and incubated in the dark at room temperature for 10 minutes, and then 100 μl of stopping solution was added to all the wells. The optical density value was determined by measurement on a flatbed spectrophotometer (Multiskan FC, Thermo) at a wavelength of 450 nm. The results of the experiment are presented in Table 2.

TABLE 2
Results of an experiment to test the expression
of various variants of the SARS-CoV-2 glycoprotein
S gene in HEK293 cells after the addition of an
immunobiological agent based on recombinant
human adenovirus of serotype 26.

| | The average value of the optical density |
|---|---|
| | at a wavelength of 450 nm |
| FSB | 0.17 (±0.08) |
| Ad26-null | 0.22 (±0.09) |
| Ad26-S-CoV-2 | 1.68 (±0.21) |
| Ad26-S-del-CoV-2 | 1.65 (0.14) |
| Ad26-S-Fc-CoV-2 | 1.71 (±0.13) |
| Ad26-RBD-CoV-2 | 1.61 (±0.18) |
| Ad26-RBD-G-CoV-2 | 1.45 (±0.22) |
| Ad26-RBD-Fc-CoV-2 | 1.51± (0.14) |

As can be seen from the data obtained, the expression of various target protein variants was observed in all cells transduced by recombinant adenoviruses pAd26-S-CoV-2, Ad26-S-del-CoV-2, Ad26-S-Fc-CoV-2, pAd26-RBD-CoV-2, pAd26-RBD-G-CoV-2, pAd26-RBD-Fc-CoV-2.

### EXAMPLE 7. A METHOD OF USING THE DEVELOPED IMMUNOBIOLOGICAL AGENT BY A SINGLE INJECTION INTO THE MAMMALIAN BODY IN AN EFFECTIVE AMOUNT FOR THE INDUCTION OF SPECIFIC IMMUNITY TO SARS-COV-2

The developed immunobiological agent based on recombinant human adenoviruses of the serotypes 5 and 26, containing a protective antigen sequence optimized for expression in mammalian cells (proteins S, S-del, S-Fc, RBD, RBD-G, RBD-Fc) SARS-CoV-2 with a sequence selected from SEQ ID NO: 1, SEQ ID NO: 2, SEQ ID NO: 3, SEQ ID NO: 4, SEQ ID NO:5, SEQ ID NO: 6 is used by introducing into the mammalian body any of the known methods of administration for this viral vector (subcutaneously, intramuscularly, intravenously, intranasally). At the same time, an immune response to the target protein of the SARS-CoV-2 glycoprotein develops in the mammalian body.

One of the main characteristics of the effectiveness of immunization is the antibody titer. The example shows data concerning changes in the titer of antibodies against the SARS-CoV-2 glycoprotein 21 days after a single intramuscular immunization of animals with an immunobiological agent, including a recombinant human adenovirus of serotype 5 or 26, containing a protective antigen sequence (proteins S, S-del, S-Fc, RBD, RBD-G, RBD-Fc) optimized for expression in mammalian cells, SARS-CoV-2 with a sequence, selected from SEQ ID NO:1, SEQ ID NO:2, SEQ ID NO:3, SEQ ID NO:4, SEQ ID NO:5, SEQ ID NO:6.

The experiment used mammals-mice of the C57BL/6 line, females 18 g. All animals were divided into 43 groups of 5 animals, which were injected intramuscularly:

1) Ad5-S-CoV-2 107BOE/mouse

2) Ad5-S-del-CoV-2 107BOE/mouse

3) Ad5-S-Fc-CoV-2 107BOE/mouse

4) Ad5-RBD-CoV-2 107BOE/mouse

5) Ad5-RBD-G-CoV-2 107BOE/mouse

6) Ad5-RBD-Fc-CoV-2 107BOE/mouse

7) Ad5-null 107BOE/mouse

8) Ad5-S-CoV-2 108BOE/mouse

9) Ad5-S-del-CoV-2 108BOE/mouse

10) Ad5-S-Fc-CoV-2 108BOE/mouse

11) Ad5-RBD-CoV-2 108BOE/mouse

12) Ad5-RBD-G-CoV-2 108BOE/mouse

13) Ad5-RBD-Fc-CoV-2 108BOE/mouse

14) Ad5-null 108BOE/mouse

15) Ad5-S-CoV-2 109BOE/mouse

16) Ad5-S-del-CoV-2 109BOE/mouse

17) Ad5-S-Fc-CoV-2 109BOE/mouse

18) Ad5-RBD-CoV-2 109BOE/mouse

19) Ad5-RBD-G-CoV-2 109BOE/mouse

20) Ad5-RBD-Fc-CoV-2 109BOE/mouse

21) Ad5-null 109BOE/mouse

22) Ad26-S-CoV-2 107BOY/mouse

23) Ad26-S-del-CoV-2 107BOE/mouse

24) Ad26-S-Fc-CoV-2 107BOE/mouse

25) Ad26-RBD-CoV-2 107BOE/mouse

26) Ad26-RBD-G-CoV-2 107BOE/mouse

27) Ad26-RBD-Fc-CoV-2 107BOE/mouse

28) Ad26-null 107BOE/mouse

29) Ad26-S-CoV-2 108BOY/mouse

30) Ad26-S-del-CoV-2 108BOE/mouse

31) Ad26-S-Fc-CoV-2 108BOE/mouse

32) Ad26-RBD-CoV-2 108BOE/mouse

33) Ad26-RBD-G-CoV-2 108BOE/mouse

34) Ad26-RBD-Fc-CoV-2 108BOE/mouse

35) Ad26-null 108BOE/mouse

36) Ad26-S-CoV-2 109BOE/mouse

37) Ad26-S-del-CoV-2 109BOE/mouse

38) Ad26-S-Fc-CoV-2 109BOE/mouse

39) Ad26-RBD-CoV-2 109BOE/mouse

40) Ad26-RBD-G-CoV-2 109BOE/mouse

41) Ad26-RBD-Fc-CoV-2 109BOE/mouse

42) Ad26-null 109BOE/mouse

43) phosphate-salt buffer

After three weeks, blood was taken from the tail vein from the animals and blood serum was isolated. The antibody titer was determined by enzyme immunoassay (ELISA) according to the following protocol:

1) Protein (S) was adsorbed on the wells of a 96-well plate for ELISA for 16 hours at a temperature of +4° C.

2) Further, to get rid of non-specific binding, the tablet was “clogged” with 5% milk dissolved in TPBS in a volume of 100 μl per well. Incubated on a shaker at a temperature of 37° C. for an hour.

3) By the method of 2-fold dilutions, serum samples of immunized mice were bred. A total of 12 dilutions of each sample were prepared.

4) 50 ml of each diluted serum sample was added to the wells of the tablet.

5) Then incubation was carried out for 1 hour at 37° C.

6) After incubation, the wells were washed three times with a phosphate buffer.

7) Then secondary antibodies against mouse immunoglobulins conjugated with horseradish peroxidase were added.

8) Then incubation was carried out for 1 hour at 37° C.

9) After incubation, the wells were washed three times with a phosphate buffer

10) Then a solution of tetramethylbenzidine (TMB) was added, which is a substrate of horseradish peroxidase and as a result of the reaction turns into a colored compound. The reaction was stopped after 15 minutes by adding sulfuric acid. Next, the optical density of the solution (OD) in each well was measured using a spectrophotometer at a wavelength of 450 nm.

The antibody titer was determined as the last dilution, in which the optical density of the solution was significantly higher than in the negative control group. The obtained results (geometric mean) are presented in Table 1.

TABLE 3
Titer of antibodies to protein S in the blood serum of mice
(geometric mean value of the antibody titer).

| Recombinant | BOE/mouse | | |
|---|---|---|---|
| adenovirus | 10⁷ | 10⁸ | 10⁹ |
| Ad5-null | 0 | 0 | 0 |
| Ad5-S-CoV-2 | 1:10809 | 1:18820 | 1:57052 |
| Ad5-S-del-CoV-2 | 1:21619 | 1:28526 | 1:114105 |
| Ad5-S-Fc-CoV-2 | 1:14263 | 1:18820 | 1:57052 |
| Ad5-RBD-CoV-2 | 1:12417 | 1:14263 | 1:99334 |
| Ad5-RBD-G-CoV-2 | 1:32768 | 1: 49667 | 1:172951 |
| Ad5-RBD-Fc-CoV-2 | 1:10809 | 1:12417 | 1:28526 |
| Ad26-null | 0 | 0 | 0 |
| Ad26-S-CoV-2 | 1:18820 | 1:24834 | 1:43238 |
| Ad26-S-del-CoV-2 | 1:24834 | 1:43238 | 1:57052 |
| Ad26-S-Fc-CoV-2 | 1:28526 | 1:32768 | 1:86475 |
| Ad26-RBD-CoV-2 | 1:12417 | 1:18820 | 1:86475 |
| Ad26-RBD-G-CoV-2 | 1:24834 | 1:32768 | 1:150562 |
| Ad26-RBD-Fc-CoV-2 | 1:9410 | 1:12417 | 1:24834 |

The results of the experiment showed that the developed immunobiological agent introduced into the mammalian body induces a humoral immune response to the SARS-CoV-2 glycoprotein in the entire range of selected doses. At the same time, it is obvious that an increase in doses will lead to an increase in the titer of antibodies in the blood of mammals before the onset of a toxic effect.

### EXAMPLE 8. A METHOD OF USING THE DEVELOPED IMMUNOBIOLOGICAL AGENT BY SEQUENTIALLY INJECTING AN EFFECTIVE AMOUNT INTO THE MAMMALIAN BODY TO INDUCE SPECIFIC IMMUNITY TO SARS-COV-2

This example describes a method for using the developed immunobiological agent based on recombinant human adenovirus of the 5th serotype, containing a sequence of protective antigen (proteins S, S-del, S-Fc, RBD, RBD-G, RBD-Fc) SARS-CoV-2 optimized for expression in mammalian cells with a sequence selected from SEQ ID NO: 1, SEQ ID NO: 2, SEQ ID NO: 3, SEQ ID NO: 4, SEQ ID NO:5, SEQ ID NO: 6 by their sequential introduction into the mammalian body at intervals of 1 week, for the induction of specific immunity to SARS-CoV-2.

The experiment was performed according to the protocol described in Example 7.

All animals were divided into 29 groups (3 animals each), which were injected intramuscularly:

1. phosphate buffer (100 mcl), and after a week a phosphate buffer (100 mcl)

2. phosphate buffer (100 μl), and after a week Ad5-null 108BOE/mouse

3. Ad5-null 108BOE/mouse, and after a week a phosphate buffer (100 mcl)

4. Ad5-null 108BOY/mouse, and a week later Ad5-null 108BOY/mouse

5. Ad5-S-CoV-2 108BOY/mouse, and a week later Ad5-S-CoV-2 108BOY/mouse

6. Ad5-S-CoV-2 108BOY/mouse, and a week later Ad5-RBD-CoV-2 108BOY/mouse

7. Ad5-S-CoV-2 108BOY/mouse, and a week later Ad5-S-del-CoV-2 108BOY/mouse

8. Ad5-S-CoV-2 108BOY/mouse, and a week later Ad5-S-Fc-CoV-2 108BOY/mouse

9. Ad5-S-CoV-2 108BOY/mouse, and a week later Ad5-RBD-CoV-2 108BOY/mouse

10. Ad5-S-CoV-2 108BOY/mouse, and a week later Ad5-RBD-G-CoV-2 108BOY/mouse

11. Ad5-S-CoV-2 108BOY/mouse, and a week later Ad5-RBD-Fc-CoV-2 108BOY/mouse

12. Ad5-S-del-CoV-2 108BOY/mouse, and a week later Ad5-S-CoV-2 108BOY/mouse

13. Ad5-S-del-CoV-2 108BOY/mouse, and a week later Ad5-S-del-CoV-2 108BOY/mouse

14. Ad5-S-del-CoV-2 108BOY/mouse, and a week later Ad5-S-Fc-CoV-2 108BOY/mouse

15. Ad5-S-del-CoV-2 108BOY/mouse, and a week later Ad5-RBD-CoV-2 108BOY/mouse

16. Ad5-S-del-CoV-2 108BOY/mouse, and a week later Ad5-RBD-G-CoV-2 108BOY/mouse

17. Ad5-S-del-CoV-2 108BOY/mouse, and a week later Ad5-RBD-Fc-CoV-2 108BOY/mouse

18. Ad5-S-Fc-CoV-2 108BOY/mouse, and a week later Ad5-S-CoV-2 108BOY/mouse

19. Ad5-S-Fc-CoV-2 108BOY/mouse, and a week later Ad5-S-del-CoV-2 108BOY/mouse

20. Ad5-S-Fc-CoV-2 108BOY/mouse, and a week later Ad5-S-Fc-CoV-2 108BOY/mouse

21. Ad5-S-Fc-CoV-2 108BOY/mouse, and a week later Ad5-RBD-CoV-2 108BOY/mouse

22. Ad5-S-Fc-CoV-2 108BOY/mouse, and a week later Ad5-RBD-G-CoV-2 108BOY/mouse

23. Ad5-S-Fc-CoV-2 108BOY/mouse, and a week later Ad5-RBD-Fc-CoV-2 108BOY/mouse

24. Ad5-RBD-CoV-2 108BOY/mouse, and a week later Ad5-S-CoV-2 108BOY/mouse

25. Ad5-RBD-CoV-2 108BOY/mouse, and a week later Ad5-S-del-CoV-2 108BOY/mouse

26. Ad5-RBD-CoV-2 108BOY/mouse, and a week later Ad5-S-Fc-CoV-2 108BOY/mouse

27. Ad5-RBD-CoV-2 108BOY/mouse, and a week later Ad5-RBD-CoV-2 108BOY/mouse

28. Ad5-RBD-CoV-2 108BOY/mouse, and a week later Ad5-RBD-G-CoV-2 108BOY/mouse

29. Ad5-RBD-CoV-2 108BOY/mouse, and a week later Ad5-RBD-Fc-CoV-2 108BOY/mouse

30. Ad5-RBD-G-CoV-2 108BOY/mouse, and a week later Ad5-S-CoV-2 108BOY/mouse

31. Ad5-RBD-G-CoV-2 108BOY/mouse, and a week later Ad5-S-del-CoV-2 108BOY/mouse

32. Ad5-RBD-G-CoV-2 108BOY/mouse, and a week later Ad5-S-Fc-CoV-2 108BOY/mouse

33. Ad5-RBD-G-CoV-2 108BOY/mouse, and a week later Ad5-RBD-CoV-2 108BOY/mouse

34. Ad5-RBD-G-CoV-2 108BOY/mouse, and a week later Ad5-RBD-G-CoV-2 108BOY/mouse

35. Ad5-RBD-G-CoV-2 108BOY/mouse, and a week later Ad5-RBD-Fc-CoV-2 108BOY/mouse

36. Ad5-RBD-Fc-CoV-2 108BOY/mouse, and a week later Ad5-S-CoV-2 108BOY/mouse

37. Ad5-RBD-Fc-CoV-2 108BOY/mouse, and a week later Ad5-S-del-CoV-2 108BOY/mouse

38. Ad5-RBD-Fc-CoV-2 108BOY/mouse, and a week later Ad5-S-Fc-CoV-2 108BOY/mouse

39. Ad5-RBD-Fc-CoV-2108BOE/mouse, and a week later Ad5-RBD-CoV-2 108BOE/mouse

40. Ad5-RBD-Fc-CoV-2 108BOY/mouse, and a week later Ad5-RBD-G-CoV-2 108BOY/mouse

41. Ad5-RBD-Fc-CoV-2 108BOY/mouse, and a week later Ad5-RBD-Fc-CoV-2 108BOY/mouse

The results are presented in tables 4 and 5.

TABLE 4
Titer of antibodies to the S protein of the SARS-CoV-2 virus
in the blood serum of mice of the control groups.

| | | Second immunization (in a week) | |
|---|---|---|---|
| | | PBS | Ad5-null |
| The first | PBS | 0 | 0 |
| immunization | Ad5-null | 0 | 0 |

TABLE 5
Titer of antibodies to the S protein of the SARS-CoV-2 virus in the blood serum
of mice of the experimental groups.

| | Second immunization (in a week) | | | | | | |
|---|---|---|---|---|---|---|---|
| | | Ad5-S- | Ad5-S- | Ad5-S- | Ad5-RBD- | Ad5-RBD- | Ad5-RBD-Fc- |
| | | CoV-2 | del-CoV-2 | Fc-CoV-2 | CoV-2 | G-CoV-2 | CoV-2 |
| First | Ad5-S- | 1:32768 | 1:131072 | 1:104032 | 1:131072 | 1:65536 | 1:104032 |
| immunization | CoV-2 | | | | | | |
| | Ad5-S-del- | 1:65536 | 1:131072 | 1:131072 | 1:131072 | 1:65536 | 1:104032 |
| | CoV-2 | | | | | | |
| | Ad5-5-Fc- | 1:65536 | 1:104032 | 1:65536 | 1:104032 | 1:52016 | 1:131072 |
| | CoV-2 | | | | | | |
| | Ad5-RBD- | 1:52016 | 1:65536 | 1:131072 | 1:65536 | 1:32768 | 1:52016 |
| | CoV-2 | | | | | | |
| | Ad5-RBD- | 1:131072 | 1:131072 | 1:104032 | 1:131072 | 1:65536 | 1:104032 |
| | G-CoV-2 | | | | | | |
| | Ad5-RBD- | 1:82570 | 1:131072 | 1:65536 | 1:32768 | 1:65536 | 1:65536 |
| | Fc-CoV-2 | | | | | | |

Thus, the results of the experiment fully confirmed that sequential immunization with the developed immunobiological agents in various combinations, including various forms of the SARS-CoV-2 protein, will cause a more powerful induction of the immune response than immunization according to a similar scheme with the same antigen.

### EXAMPLE 9. DETERMINATION OF THE EFFECTIVENESS OF IMMUNIZATION BY THE DEVELOPED IMMUNOBIOLOGICAL TOOL FOR ASSESSING THE PROPORTION OF PROLIFERATING LYMPHOCYTES

Proliferative analysis allows us to assess the ability of lymphocytes to divide intensively after meeting with an antigen. In order to assess the proliferation, the authors used the staining of lymphocytes with a fluorescent dye CFSE. This dye binds to cellular proteins, and persists for a long time and is never transmitted to neighboring cells in the population. However, the fluorescent label is transmitted to the daughter cells. The concentration of the label, and, consequently, the intensity of fluorescence, decreases exactly twice. Therefore, dividing cells are easy to track by reducing their fluorescence.

Mice of the C57BL/6 line were used in the experiment. All animals were divided into 8 groups (3 animals each), which were injected intramuscularly:

1) phosphate buffer (100 μl)

2) Ad5-null 108BOE/mouse

3) Ad5-S-CoV-2 108BOE/mouse

4) Ad5-S-del-CoV-2 108BOE/mouse

5) Ad5-S-Fc-CoV-2 108BOE/mouse

6) Ad5-RBD-CoV-2 108BOE/mouse

7) Ad5-RBD-G-CoV-2 108BOE/mouse

8) Ad5-RBD-Fc-CoV-2 108BOE/mouse

The doses of recombinant adenoviruses were selected based on the data obtained during the determination of the antibody titer.

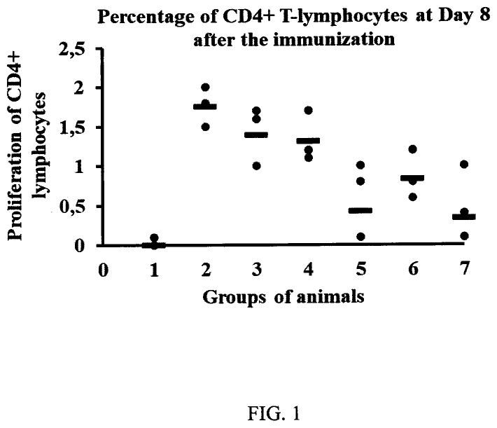

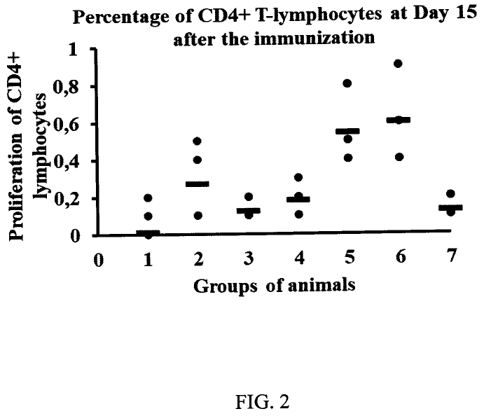

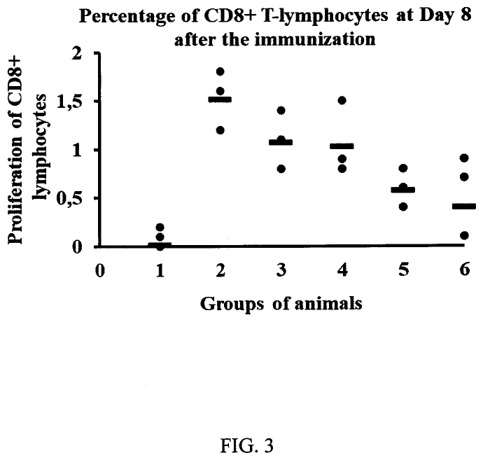

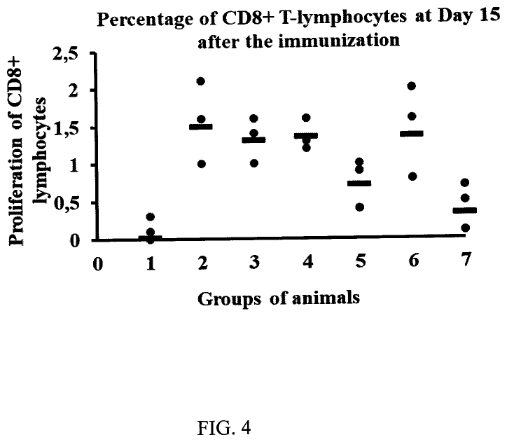

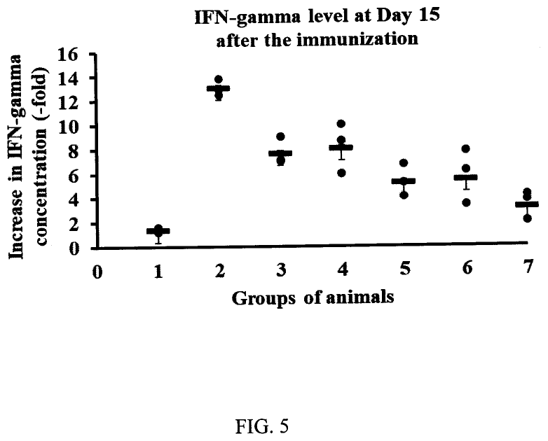

After two weeks, the animals were euthanized. Lymphocytes were isolated from the spleen by centrifugation in the ficoll-urographin gradient. Then the isolated cells were stained with CFSE according to the method (B. J. Quah et. al., Monitoring lymphocyte proliferation in vitro and in vivo with the intracellular fluorescent dye carboxyfluorescein diacetate succinimidyl ester, Nature Protocols, 2007, X2 2(9), 2049-2056) and cultured in the presence of the antigen. Next, the cells were analyzed by flow cytofluorimetry. The results obtained are shown in FIGS. 1, 2, 3, 4. Thus, it can be concluded that the obtained adenoviral constructs induce the formation of an antigen-specific immune response (both CD4+ and CD8+).

As can be seen from the results of the experiment (FIG. 1, 2, 3, 4), the developed immunobiological agents according to claim 1, claim 2, claim 3, claim 4, claim 5 at this dose effectively stimulate the proliferation of lymphocytes.

### EXAMPLE 10. A METHOD OF USING THE DEVELOPED IMMUNOBIOLOGICAL AGENTS BASED ON RECOMBINANT HUMAN ADENOVIRUSES OF THE 5TH AND 26TH SEROTYPES BY THEIR SEQUENTIAL INTRODUCTION INTO THE MAMMALIAN BODY TO INDUCE SPECIFIC IMMUNITY TO SARS-COV-2

The experiment was performed according to the protocol described in Example 7. Combinations of immunobiological agents were selected based on Examples 7 and 8.

All animals were divided into 31 groups (3 animals each), which were injected intramuscularly:

1. phosphate buffer (100 mcl), and after a week a phosphate buffer (100 mcl)

2. Ad26-null 108BOE/mouse, and after a week a phosphate buffer (100 μl)

3. phosphate buffer (100 μl), and after a week Ad26-null 108BOE/mouse

4. Ad26-null 108BOY/mouse, and a week later Ad26-null 108BOY/mouse

5. Ad5-null 108BOE/mouse, and after a week a phosphate buffer (100 μl)

6. phosphate buffer (100 μl), and after a week Ad5-null 108BOE/mouse

7. Ad5-null 108BOY/mouse, and a week later Ad5-null 108BOY/mouse

8. Ad5-null 108BOY/mouse, and a week later Ad26-null 108BOY/mouse

9. Ad26-null 108BOY/mouse, and a week later Ad5-null 108BOY/mouse

10. Ad5-S-CoV-2 108BOY/mouse, and a week later Ad26-RBD-G-CoV-2 108BOY/mouse

11. Ad5-RBD-G-CoV-2 108BOY/mouse, and a week later Ad26-S-CoV-2 108BOY/mouse

12. Ad26-S-CoV-2 108BOY/mouse, and a week later Ad5-RBD-G-CoV-2 108BOY/mouse

13. Ad26-RBD-G-CoV-2 108BOY/mouse, and a week later Ad5-S-CoV-2 108BOY/mouse

14. Ad5-S-del-CoV-2 108BOY/mouse, and a week later Ad26-RBD-CoV-2 108BOY/mouse

15. Ad26-S-G-CoV-2 108BOY/mouse, and a week later Ad5-RBD-CoV-2 108BOY/mouse

16. Ad5-RBD-CoV-2 108BOY/mouse, and a week later Ad26-S-del-CoV-2 108BOY/mouse

17. Ad26-S-G-CoV-2 108BOY/mouse, and a week later Ad5-RBD-CoV-2 108BOY/mouse

18. Ad26-RBD-CoV-2 108BOY/mouse, and a week later Ad5-S-del-CoV-2 108BOY/mouse

19. Ad5-RBD-CoV-2 108BOY/mouse, and a week later Ad26-S-del-CoV-2 108BOY/mouse

20. Ad5-S-del-CoV-2 108BOY/mouse, and a week later Ad26-RBD-G-CoV-2 108BOY/mouse

21. Ad5-RBD-G-CoV-2 108BOY/mouse, and a week later Ad26-S-del-CoV-2 108BOY/mouse

22. Ad26-S-del-CoV-2 108BOY/mouse, and a week later Ad5-RBD-G-CoV-2 108BOY/mouse

23. Ad26-RBD-G-CoV-2 108BOY/mouse, and a week later Ad5-S-G-CoV-2 108BOY/mouse

24. Ad5-S-CoV-2 108BOY/mouse, and a week later Ad26-RBD-CoV-2 108BOY/mouse

25. Ad26-S-CoV-2 108BOY/mouse, and a week later Ad5-RBD-CoV-2 108BOY/mouse

26. Ad5-RBD-CoV-2 108BOY/mouse, and a week later Ad26-S-CoV-2 108BOY/mouse

27. Ad26-RBD-CoV-2 108BOY/mouse, and a week later Ad5-S-CoV-2 108BOY/mouse

28. Ad5-S-del-CoV-2 108BOY/mouse, and a week later Ad26-S-CoV-2 108BOY/mouse

29. Ad26-S-del-CoV-2 108BOY/mouse, and a week later Ad5-S-CoV-2 108BOY/mouse

30. Ad5-S-CoV-2 108BOY/mouse, and a week later Ad26-S-del-CoV-2 108BOY/mouse

31. Ad26-S-CoV-2 108BOY/mouse, and a week later Ad5-S-del-CoV-2 108BOY/mouse

The results are presented in tables 6 and 7.

TABLE 6
Titer of antibodies to the S protein of the SARS-CoV-2
virus in the blood serum of mice of the control groups.

| | | Second immunization (in a week) | | |
|---|---|---|---|---|
| | | PBS | Ad5-null | Ad26-null |
| First | PBS | 0 | 0 | 0 |
| imminization | Ad5-null | 0 | 0 | 0 |
| | Ad26-null | 0 | 0 | 0 |

TABLE 7
Titer of antibodies to the S protein of the SARS-CoV-2 virus
in the blood serum of mice of the experimental groups.

| | The geometric mean value |
|---|---|
| Group of animals | of the antibody titer |
| Ad5-S-CoV-2 108BOE/mouse, and a week | 1:208064 |
| later Ad26-RBD-G 108BOE/mouse | |
| Ad5-RBD-G-CoV-2 108BOY/mouse, and a | 1:1321123 |
| week later Ad26-S-CoV-2 108BOY/mouse | |
| Ad26-S-CoV-2 108BOY/mouse, and a week | 1:832255 |
| later Ad5-RBD-G-CoV-2 108BOY/mouse | |
| Ad26-RBD-G-CoV-2 108BOY/mouse, and a | 1:1321123 |
| week later Ad5-S-CoV-2 108BOY/mouse | |
| Ad5-S-del-CoV-2 108BOY/mouse, and a | 1:165140 |
| week later Ad26-RBD-CoV-2 108BOY/mouse | |
| Ad26-S-del-CoV-2 108BOY/mouse, and a | 1:104032 |
| week later Ad5-RBD-CoV-2 108BOY/mouse | |
| Ad5-RBD-CoV-2 108BOY/mouse, and a | 1:104032 |
| week later Ad26-S-del-CoV-2 108BOY/mouse | |
| Ad26-S-del-CoV-2 108BOY/mouse, and a | 1:52016 |
| week later Ad5-RBD-CoV-2 108BOY/mouse | |
| Ad26-RBD-CoV-2 108BOY/mouse, and a | 1:131072 |
| week later Ad5-S-del-CoV-2 108BOY/mouse | |
| Ad5-RBD-CoV-2 108BOY/mouse, and a | 1:104032 |
| week later Ad26-S-del-CoV-2 108BOY/mouse | |
| Ad5-S-del-CoV-2 108BOY/mouse, and a | 1:165140 |
| week later Ad26-RBD-G-CoV-2 | |
| 108BOY/mouse | |
| Ad5-RBD-G-CoV-2 108BOY/mouse, and a | 1:208064 |
| week later Ad26-S-del-CoV-2 108BOY/mouse | |
| Ad26-S-del-CoV-2 108BOY/mouse, and a | 1:660561 |
| week later Ad5-RBD-G-CoV-2 | |
| 108BOY/mouse | |
| Ad26-RBD-G-CoV-2 108BOY/mouse, and a | 1:416128 |
| week later Ad5-S-del-CoV-2 108BOY/mouse | |
| Ad5-S-CoV-2 108BOY/mouse, and a week | 1:208064 |
| later Ad26-RBD-CoV-2 108BOY/mouse | |
| Ad26-S-CoV-2 108BOY/mouse, and a week | 1:65536 |
| later Ad5-RBD-CoV-2 108BOY/mouse | |
| Ad5-RBD-CoV-2 108BOY/mouse, and a | 1:131072 |
| week later Ad26-S-CoV-2 108BOY/mouse | |
| Ad26-RBD-CoV-2 108BOY/mouse, and a | 1:165140 |
| week later Ad5-S-CoV-2 108BOY/mouse | |
| Ad5-S-del-CoV-2 108BOY/mouse, and a | 1:208064 |
| week later Ad26-S-CoV-2 108BOY/mouse | |
| Ad26-S-G-CoV-2 108BOY/mouse, and a | 1:208064 |
| week later Ad5-S-CoV-2 108BOY/mouse | |
| Ad5-S-CoV-2 108BOY/mouse, and a week | 1:165140 |
| later Ad26-S-del-CoV-2 108BOY/mouse | |
| Ad26-S-CoV-2 108BOY/mouse, and a week | 1:165140 |
| later Ad5-S-del-CoV-2 108BOY/mouse | |

Thus, the results of the experiment fully confirmed that sequential immunization with developed immunobiological agents including various adenovirus vectors (based on human adenoviruses of serotypes 5 and 26) will cause a more powerful induction of the immune response than immunization according to a similar scheme with the same vector.

### EXAMPLE 11. DETERMINATION OF THE EFFECTIVENESS OF IMMUNIZATION BY THE DEVELOPED IMMUNOBIOLOGICAL TOOL FOR ASSESSING THE INDUCTION OF IFN-GAMMA

In this experiment, the effectiveness of immunization was evaluated by a developed immunobiological agent based on a recombinant adenovirus containing a protective antigen sequence (proteins S, S-del, S-Fc, RBD, RBD-G, RBD-Fc) of the SARS-COV-2 virus optimized for expression in mammalian cells with a sequence selected from SEQ ID NO: 1, SEQ ID NO: 2, SEQ ID NO: 3, SEQ ID NO: 4, SEQ ID NO: 5, SEQ ID NO:6 to determine the increase in the concentration of IFN-gamma in the medium after stimulation of splenocytes of C57/BL6 mice immunized with adenovirus constructs with a recombinant full-size S protein of the SARS-CoV-2 virus.

To determine the level of IFN-gamma, the Mouse IFN gamma Platinum ELISA kit (Affymetrix eBioscience, USA) was used.

The procedure for conducting the IFA. The wells of the tablet were washed with a single buffer for washing twice with a volume of 200 μl per well, and then 100 μl of standards and 100 μl of sample dilution solution were added as a negative control. 50 ml of dilution solution was added to the sample wells, and then 50 ml of the samples themselves (medium from stimulated splenocytes). A solution of antibodies conjugated with biotin was prepared. To do this, a conjugate with a volume of 60 μl was diluted in 5.94 ml of buffer for analysis. Then, 50 ml of a solution of antibodies conjugated with biotin was added to all the wells, the tablet was covered with a lid and incubated at room temperature on a shaker at 400 rpm for 2 hours. Next, a solution of streptavidin conjugated with horseradish peroxidase was prepared. To do this, a conjugate with a volume of 60 μl was diluted in 5.94 ml of buffer for analysis. The wells of the tablet were washed twice with a single buffer for washing with a volume of 200 ml per well and 100 ml of streptavidin solution conjugated with horseradish peroxidase was added to all the wells of the tablet. The tablet was incubated at room temperature on a shaker at 400 revolutions per minute for 1 hour. Then the wells of the tablet were washed twice with a single buffer for washing with a volume of 200 μl per well and 100 μl of TMB of substrate was added to all the wells of the tablet and incubated in the dark at room temperature for 10 minutes, and then 100 μl of stopping solution was added to all the wells. The optical density value was determined by measurement on a flatbed spectrophotometer (Multiskan FC, Thermo) at a wavelength of 450 nm.

The results of measuring the production of IFN-gamma on the 15th day after immunization of the test animals with adenovirus constructs are shown graphically in FIG. 5 in the form of an increase in the concentration of IFN-gamma (times) when comparing cells stimulated by the recombinant full-size protein S of the SARS-CoV-2 virus with intact cells.

According to the results of the study, it was shown that the introduction of the obtained structures to animals leads to a high level of induction of IFN-gamma expression by splenocytes when stimulated by the recombinant S protein of the SARS-CoV-2 virus, which indicates the formation of specific T-cell immunity.

### EXAMPLE 12. A METHOD FOR USING THE DEVELOPED IMMUNOBIOLOGICAL AGENTS BASED ON RECOMBINANT HUMAN ADENOVIRUSES OF THE 5TH SEROTYPE CONTAINING A SEQUENCE OF PROTECTIVE ANTIGEN (PROTEIN S AND RBD-G) SARS-COV-2 OPTIMIZED FOR EXPRESSION IN MAMMALIAN CELLS WITH A SEQUENCE SELECTED FROM SEQ ID NO: 1 AND SEQ ID NO: 5 BY THEIR SIMULTANEOUS INTRODUCTION INTO THE MAMMALIAN BODY TO INDUCE SPECIFIC IMMUNITY TO SARS-COV-2

The experiment was performed according to the protocol described in Example 7. The combination of immunobiological agents was selected based on Examples 8 and 11.

All animals were divided into 17 groups (5 animals each), which were injected intramuscularly:

1. phosphate buffer (100 ml)

2. Ad5-null 105 virus particles/mouse

3. Ad5-null 106 virus particles/mouse

4. Ad5-null 107 virus particles/mouse

5. Ad5-null 108 virus particles/mouse

6. Ad5-null 109 virus particles/mouse

7. Ad5-null 1010 virus particles/mouse

8. Ad5-null 5*1010 virus particles/mouse

9. Ad5-null 1011 virus particles/mouse

10. Ad5-S-CoV-2+Ad5-RBD-G-CoV-2 105 virus particles/mouse

11. Ad5-S-CoV-2+Ad5-RBD-G-CoV-2 106 virus particles/mouse

12. Ad5-S-CoV-2+Ad5-RBD-G-CoV-2 107 virus particles/mouse

13. Ad5-S-CoV-2+Ad5-RBD-G-CoV-2 108 virus particles/mouse

14. Ad5-S-CoV-2+Ad5-RBD-G-CoV-2 109 virus particles/mouse

15. Ad5-S-CoV-2+Ad5-RBD-G-CoV-2 1010 virus particles/mouse

16. Ad5-S-CoV-2+Ad5-RBD-G-CoV-2 5*1010 virus particles/mouse

17. Ad5-S-CoV-2+Ad5-RBD-G-CoV-2 1011 virus particles/mouse

The results are presented in tables 8 and 9.

TABLE 8
Titer of antibodies to the S protein of the SARS-CoV-2 virus
in the blood serum of mice of the control groups.

| | The geometric mean value of the titer |
|---|---|
| | of antibodies to the S SARS-CoV-2 |
| Group of animals | protein |
| phosphate buffer | 0 |
| Ad5-null 10⁵ virus particles/mouse | 0 |
| Ad5-null 10⁶ virus particles/mouse | 0 |
| Ad5-null 10⁷ virus particles/mouse | 0 |
| Ad5-null 10⁸ virus particles/mouse | 0 |
| Ad5-null 10⁹ virus particles/mouse | 0 |
| Ad5-null 10¹⁰ virus particles/mouse | 0 |
| Ad5-null 5*10¹⁰ virus particles/mouse | 0 |
| Ad5-null 10¹¹ virus particles/mouse | 0 |

TABLE 9
Titer of antibodies to the S protein of the SARS-CoV-2
virus in the blood serum of mice of the experimental groups.

| | The geometric mean |
|---|---|
| | value of the titer of |
| | antibodies to the S |
| Group of animals, virus particles/mouse | SARS-CoV-2 protein |
| Ad5-S-CoV-2 + Ad5-RBD-G-CoV-2 10⁵ | 0 |
| Ad5-S-CoV-2 + Ad5-RBD-G-CoV-2 10⁶ | 1:14263 |
| Ad5-S-CoV-2 + Ad5-RBD-G-CoV-2 10⁷ | 1:99334 |
| Ad5-S-CoV-2 + Ad5-RBD-G-CoV-2 10⁸ | 1:131072 |
| Ad5-S-CoV-2 + Ad5-RBD-G-CoV-2 10⁹ | 1:172951 |
| Ad5-S-CoV-2 + Ad5-RBD-G-CoV-2 10¹⁰ | 1:301124 |
| Ad5-S-CoV-2 + Ad5-RBD-G-CoV-2 5*10¹⁰ | 1:345901 |
| Ad5-S-CoV-2 + Ad5-RBD-G-CoV-2 10¹¹ | 1:524288 |

Thus, the results of the experiment fully confirmed that simultaneous immunization with the developed immunobiological agents induces a humoral immune response to the SARS-CoV-2 glycoprotein in the dose range from 106 viral particles/mouse to 1011 viral particles/mouse. At the same time, it is obvious that an increase in doses will lead to an increase in the titer of antibodies in the blood of mammals before the onset of a toxic effect.

### EXAMPLE 13 A METHOD OF USING THE DEVELOPED IMMUNOBIOLOGICAL AGENT BY SEQUENTIALLY INJECTING INTO THE MAMMALIAN BODY AT VARIOUS TIME INTERVALS IN AN EFFECTIVE AMOUNT TO INDUCE SPECIFIC IMMUNITY TO SARS-COV-2

This example describes a method for using the developed immunobiological agent based on recombinant human adenovirus of the 5th serotype, containing a sequence of protective antigen (proteins S, S-del, RBD, RBD-G, RBD-Fc) SARS-CoV-2 optimized for expression in mammalian cells with a sequence selected from SEQ ID NO: 1, SEQ ID NO: 2, SEQ ID NO: 3, SEQ ID NO: 4, SEQ ID NO:5, SEQ ID NO: 6 by their sequential introduction into the mammalian body at intervals of 1 week or at intervals of 3 weeks to induce specific immunity to SARS-CoV-2.

The experiment was performed according to the protocol described in Example 7.

All animals were divided into 28 groups (3 animals each), which were injected intramuscularly:

1. phosphate buffer (100 mcl), and after a week a phosphate buffer (100 mcl)

2. Ad5-null 108BOY/mouse, and a week later Ad5-null 108BOY/mouse

3. Ad5-S-CoV-2 108BOY/mouse, and a week later Ad5-S-CoV-2 108BOY/mouse

4. Ad5-S-del-CoV-2 108BOY/mouse, and a week later Ad5-S-del-CoV-2 108BOY/mouse

5. Ad5-S-Fc-CoV-2 108BOY/mouse, and a week later Ad5-S-Fc-CoV-2 108BOY/mouse

6. Ad5-RBD-CoV-2 108BOY/mouse, and a week later Ad5-RBD-CoV-2 108BOY/mouse

7. Ad5-RBD-G-CoV-2 108BOY/mouse, and a week later Ad5-RBD-G-CoV-2 108BOY/mouse

8. Ad5-RBD-Fc-CoV-2 108BOY/mouse, and a week later Ad5-RBD-Fc-CoV-2 108BOY/mouse

9. Ad26-S-CoV-2 108BOY/mouse, and a week later Ad26-S-CoV-2 108BOY/mouse

10. Ad26-S-del-CoV-2 108BOY/mouse, and a week later Ad26-S-del-CoV-2 108BOY/mouse

11. Ad26-S-Fc-CoV-2 108BOY/mouse, and a week later Ad26-S-Fc-CoV-2 108BOY/mouse

12. Ad26-RBD-CoV-2 108BOY/mouse, and a week later Ad26-RBD-CoV-2 108BOY/mouse

13. Ad26-RBD-G-CoV-2 108BOY/mouse, and a week later Ad26-RBD-G-CoV-2 108BOY/mouse

14. Ad26-RBD-Fc-CoV-2 108BOY/mouse, and a week later Ad26-RBD-Fc-CoV-2 108BOY/mouse

15. phosphate buffer (100 mcl), and after 3 weeks phosphate buffer (100 mcl)

16. Ad5-null 108BOY/mouse, and after 3 weeks Ad5-null 108BOY/mouse

17. Ad5-S-CoV-2 108BOY/mouse, and after 3 weeks Ad5-S-CoV-2 108BOY/mouse

18. Ad5-S-del-CoV-2 108BOY/mouse, and after 3 weeks Ad5-S-del-CoV-2 108BOY/mouse

19. Ad5-S-Fc-CoV-2 108BOY/mouse, and after 3 weeks Ad5-S-Fc-CoV-2 108BOY/mouse

20. Ad5-RBD-CoV-2 108BOY/mouse, and after 3 weeks Ad5-RBD-CoV-2 108BOY/mouse

21. Ad5-RBD-G-CoV-2 108BOY/mouse, and after 3 weeks Ad5-RBD-G-CoV-2 108BOY/mouse

22. Ad5-RBD-Fc-CoV-2 108BOY/mouse, and after 3 weeks Ad5-RBD-Fc-CoV-2 108BOY/mouse

23. Ad26-S-CoV-2 108BOY/mouse, and after 3 weeks Ad26-S-CoV-2 108BOY/mouse

24. Ad26-S-del-CoV-2 108BOY/mouse, and after 3 weeks Ad26-S-del-CoV-2 108BOY/mouse

25. Ad26-S-Fc-CoV-2 108BOY/mouse, and after 3 weeks Ad26-S-Fc-CoV-2 108BOY/mouse

26. Ad26-RBD-CoV-2 108BOY/mouse, and after 3 weeks Ad26-RBD-CoV-2 108BOY/mouse

27. Ad26-RBD-G-CoV-2 108BOY/mouse, and after 3 weeks Ad26-RBD-G-CoV-2 108BOY/mouse

28. Ad26-RBD-Fc-CoV-2 108BOY/mouse, and after 3 weeks Ad26-RBD-Fc-CoV-2 108BOY/mouse

TABLE 10
Titer of antibodies to the S protein of the SARS-CoV-2
virus in the blood serum of mice.

| | Second immunization | |
|---|---|---|
| | with an interval | with an interval |
| | of 1 week | of 2 weeks |
| FSB/FSB | 0 | 0 |
| Ad5-null/Ad5-null | 0 | 0 |
| Ad5-S-CoV-2/Ad5-S-CoV- | 1:32768 | 1:41285 |
| 2 | | |
| Ad5-S-del-CoV-2/Ad5-S- | 1:41285 | 1:52016 |
| del-CoV-2 | | |
| Ad5-S-Fc-CoV-2/Ad5-S- | 1:82570 | 1:104032 |
| Fc-CoV-2 | | |
| Ad5-RBD-CoV-2/Ad5- | 1:65536 | 1:82570 |
| RBD-CoV-2 | | |
| Ad5-RBD-G-CoV-2/Ad5- | 1:65536 | 1:82570 |
| RBD-G-CoV-2 | | |
| Ad5-RBD-Fc-CoV-2/Ad5- | 1:65536 | 1:104032 |
| RBD-Fc-CoV-2 | | |
| Ad26-S-CoV-2/Ad26-S- | 1:26008 | 1:32768 |
| CoV-2 | | |
| Ad26-S-del-CoV-2/Ad26- | 1:52016 | 1:65536 |
| S-del-CoV-2 | | |
| Ad26-S-Fc-CoV-2/Ad26-S- | 1:26008 | 1:52016 |
| Fc-CoV-2 | | |
| Ad26-RBD-CoV-2/Ad26- | 1:20643 | 1:26008 |
| RBD-CoV-2 | | |
| Ad26-RBD-G-CoV-2/ | 1:41285 | 1:52016 |
| Ad26-RBD-G-CoV-2 | | |
| Ad26-RBD-Fc-CoV-2/ | 1:13004 | 1:16384 |
| Ad26-RBD-Fc-CoV-2 | | |

Thus, the results of the experiment confirm that sequential immunization with the developed immunobiological agent leads to higher levels of immune response, compared with a single immunization. It is obvious to a mid-level specialist that the final scheme of immunization with a ready-made drug is based on many years of research and is often adjusted directly by a doctor, it depends on many factors, including the target group of patients, their age, the epidemiological situation, etc.

### EXAMPLE 14 A METHOD OF USING THE DEVELOPED IMMUNOBIOLOGICAL AGENT BY SEQUENTIALLY INJECTING INTO THE MAMMALIAN BODY AT INTERVALS OF ONE WEEK IN AN EFFECTIVE AMOUNT TO INDUCE SPECIFIC IMMUNITY TO SARS-COV-2

This example describes a method for using the developed immunobiological agent based on recombinant human adenovirus of the 5th serotype and recombinant human adenovirus of the 26th serotype, by their sequential introduction into the mammalian body at intervals of 1 week to induce specific immunity to SARS-CoV-2.

The experiment was performed according to the protocol described in Example 7.

All animals were divided into 9 groups (5 animals each), which were injected intramuscularly:

1. phosphate buffer (100 mcl), then a week later a phosphate buffer (100 mcl), then a week later a phosphate buffer (100 mcl)

2. Ad5-null 108BOY/mouse, then a week later Ad5-null 108BOY/mouse, then a week later Ad5-null 108BOY/mouse

3. Ad26-null 108BOY/mouse, then a week later Ad26-null 108BOY/mouse, then a week later Ad26-null 108BOY/mouse

4. Ad5-S-CoV-2 108BOY/mouse, then a week later Ad5-S-CoV-2 108BOY/mouse, then a week later Ad5-S-CoV-2 108BOY/mouse

5. Ad5-S-CoV-2 108BOY/mouse, then a week later Ad26-S-CoV-2 108BOY/mouse, then a week later Ad5-S-CoV-2 108BOY/mouse

6. Ad5-S-CoV-2 108BOY/mouse, then a week later Ad26-S-CoV-2 108BOY/mouse, then a week later Ad26-S-CoV-2 108BOY/mouse

7. Ad26-S-CoV-2 108BOY/mouse, then a week later Ad26-S-CoV-2 108BOY/mouse, then a week later Ad26-S-CoV-2 108BOY/mouse

8. Ad26-S-CoV-2 108BOY/mouse, then a week later Ad5-S-CoV-2 108BOY/mouse, then a week later Ad26-S-CoV-2 108BOY/mouse

9. Ad26-S-CoV-2 108BOY/mouse, then a week later Ad5-S-CoV-2 108BOY/mouse, then a week later Ad5-S-CoV-2 108BOY/mouse

TABLE 11
Titer of antibodies to the S protein of the SARS-CoV-2 virus
in the blood serum of mice

| Group of animals | Antibody titer |
|---|---|
| FSB/FSB/FSB | 0 |
| Ad5-null/Ad5-null/Ad5-null | 0 |
| Ad26-null/Ad26-null/Ad26-null | 0 |
| Ad5-S-CoV-2/Ad5-S-CoV-2/Ad5-S-CoV-2 | 1:150562 |
| Ad5-S-CoV-2/Ad26-S-CoV-2/Ad5-S-CoV-2 | 1:301124 |
| Ad5-S-CoV-2/Ad26-S-CoV-2/Ad26-S-CoV-2 | 1:228209 |
| Ad26-S-CoV-2/Ad26-S-CoV-2/Ad26-S-CoV-2 | 1:172950 |
| Ad26-S-CoV-2/Ad5-S-C oV-2/Ad26-S-CoV-2 | 1:262144 |
| Ad26-S-CoV-2/Ad5-S-CoV-2/Ad5-S-CoV-2 | 1:301124 |

Thus, the results of this experiment on the example of the developed immunobiological agent based on recombinant human adenovirus of serotype 5 or 26, containing the SARS-CoV-2 protein sequence optimized for expression in mammalian cells, showed that 3-fold sequential administration of any variants of this agent leads to a stronger immune response to the antigen, compared with a single and double administration. It is obvious to a mid-level specialist that the developed immunobiological agent can be administered repeatedly, which will lead to an increase in the titer of antibodies in the blood of mammals before the onset of a toxic effect. The required number of immunizations may differ depending on the target population category (their nationality, age, work, etc.). The frequency of immunization is also determined by economic feasibility.

### EXAMPLE 15 A METHOD OF USING THE DEVELOPED IMMUNOBIOLOGICAL AGENT BY A SINGLE INJECTION INTO THE MAMMALIAN BODY IN VARIOUS WAYS IN AN EFFECTIVE AMOUNT TO INDUCE SPECIFIC IMMUNITY TO SARS-COV-2

This example describes a method of using the developed immunobiological agent based on recombinant human adenovirus of the 5th serotype and recombinant human adenovirus of the 26th serotype, by their single introduction into the mammalian body by 3 methods (intranasal, subcutaneous, intramuscular) to induce specific immunity to SARS-CoV-2.

The experiment was performed according to the protocol described in Example 7.

All animals were divided into 15 groups (3 animals each), which were injected:

1. FSB intranasally

2. FSB subcutaneously

3. FSB intramuscularly

4. Ad5-null 109BOE/mouse intranasally

5. Ad5-null 109BOE/mouse subcutaneously

6. Ad5-null 109BOY/mouse intramuscularly

7. Ad26-null 109BOY/mouse intranasally

8. Ad26-null 109BOE/mouse subcutaneously

9. Ad26-null 109BOE/mouse intramuscularly

10. Ad5-S-CoV-2 109BOE/mouse intranasally

11. Ad5-S-CoV-2 109BOE/mouse subcutaneously

12. Ad5-S-CoV-2 109BOE/mouse intramuscularly

13. Ad26-S-CoV-2 109BOE/mouse intranasally

14. Ad26-S-CoV-2 109BOE/mouse subcutaneously

15. Ad26-S-CoV-2 109BOE/mouse intramuscularly

The results are presented in table 12.

TABLE 12
Titer of antibodies to the S protein of the SARS-CoV-2 virus
in the blood serum of mice

| Group of animals | Antibody titer |
|---|---|
| FSB intranasally | 0 |
| FSB subcutaneously | 0 |
| FSB intramuscularly | 0 |
| Ad5-null intranasally | 0 |
| Ad5-null subcutaneously | 0 |
| Ad5-null intramuscularly | 0 |
| Ad26-nullintranasally | 0 |
| Ad26-null subcutaneously | 0 |
| Ad26-null [symbol] | 0 |
| Ad5-S-CoV-2 intranasally | 1:16384 |
| Ad5-S-CoV-2 subcutaneously | 1:26008 |
| Ad5-S-CoV-2 intramuscularly | 1:57052 |
| Ad26-S-CoV-2 intranasally | 1:13004 |
| Ad26-S-CoV-2 subcutaneously | 1:24300 |
| Ad26-S-CoV-2 intramuscularly | 1:43238 |

Thus, the results of this experiment confirm the possibility of using the developed immunobiological agent for the induction of specific immunity to the SARS-CoV-2 virus by its intranasal, intramuscular or subcutaneous administration.

### INDUSTRIAL APPLICABILITY

The advantage of the claimed technical solution is the use of such doses of recombinant adenoviruses expressing the full-size protein gene, which can increase immunogenicity, but at which toxic effects on animals are not yet observed. Also, the advantages include an additional increase in the immunogenicity of the receptor-binding domain of the S gene of the SARS-CoV-2 virus due to the addition of a leader sequence for protein secretion from the cell to the external environment. One of the advantages of the claimed technical solution is the presence of an adequate T-cell response (both CD4+ and CD8+) to the introduction of the antigen.

Thus, an immunobiological agent based on recombinant human adenovirus of the 5th serotype was created, containing human adenovirus of the 5th serotype with deleted E1/E3 regions and an embedded genetic construct encoding the developed optimal amino acid sequences of the protective antigen S of the SARS-CoV-2 virus.

Also, an immunobiological agent based on recombinant human adenovirus of the 26th serotype was created, containing human adenovirus of serotype 26 with deleted E1/E3 regions, replaced by an open reading frame 6 with an open reading frame of human adenovirus of the serotype 5 and with an integrated genetic construct encoding the developed optimal amino acid sequences of the protective antigen S of the SARS-CoV-2 virus. In this case, the coding sequences of various forms of the S protein of the SARS-CoV-2 virus are expressed by recombinant pseudoadenovirus particles directly in the subject's body.

The developed immunobiological agent can be considered as a drug for preclinical studies, as an antiviral vaccine that can effectively protect a person from infection with the SARS-CoV-2 coronavirus. The technology of production of such a vaccine is proposed.

1. Immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of S protective antigen of the SARS-CoV-2 virus with gene C′-terminal deletion of 18 amino acids (SEQ ID NO:2).

2. Immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of full-length S protective antigen of the SARS-CoV-2 virus and the human IgG1 Fc-fragment sequence (SEQ ID NO:3).

3. Immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus S protein receptor-binding domain sequence with the viral leader peptide sequence (SEQ ID NO:4).

4. Immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus protein S receptor-binding domain sequence with the transmembrane domain of vesicular stomatitis virus glycoprotein (SEQ ID NO:5).

5. Immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus S protein receptor-binding domain sequence with the leader peptide sequence and the human IgG1 Fc-fragment sequence (SEQ ID NO:6).

6. Immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus full-length S protective antigen sequence on the basis of sequences of S protein genes of the SARS-CoV-2 virus (SEQ ID NO:1) in combination with one or more immunobiological agents selected from a group consisting of
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of S protective antigen of the SARS-CoV-2 virus with gene C′-terminal deletion of 18 amino acids (SEQ ID NO:2);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of full-length S protective antigen of the SARS-CoV-2 virus and the human IgG1 Fc-fragment sequence (SEQ ID NO:3);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus S protein receptor-binding domain sequence with the viral leader peptide sequence (SEQ ID NO:4);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus protein S receptor-binding domain sequence with the transmembrane domain of vesicular stomatitis virus glycoprotein (SEQ ID NO:5);
an immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus S protein receptor-binding domain sequence with the leader peptide sequence and the human IgG1 Fc-fragment sequence (SEQ ID NO:6); and
a second immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus full-length S protective antigen sequence on the basis of sequences of S protein genes of the SARS-CoV-2 virus (SEQ ID NO:1).

7. Method of induction of specific immunity to the SARS-CoV-2 virus, involving the administration to mammals of one or more immunobiological agents selected from a group consisting of
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of S protective antigen of the SARS-CoV-2 virus with gene C′-terminal deletion of 18 amino acids (SEQ ID NO:2);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of full-length S protective antigen of the SARS-CoV-2 virus and the human IgG1 Fc-fragment sequence (SEQ ID NO:3);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus S protein receptor-binding domain sequence with the viral leader peptide sequence (SEQ ID NO:4);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus protein S receptor-binding domain sequence with the transmembrane domain of vesicular stomatitis virus glycoprotein (SEQ ID NO:5);
an immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus S protein receptor-binding domain sequence with the leader peptide sequence and the human IgG1 Fc-fragment sequence (SEQ ID NO:6); and
an immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus full-length S protective antigen sequence on the basis of sequences of S protein genes of the SARS-CoV-2 virus (SEQ ID NO:1),
in an effective amount.

8. Method presented herein in claim 7, wherein two or more different immunobiological agents based on recombinant human adenovirus serotype 5 or two or more different immunobiological agents based on recombinant human adenovirus serotype 26 selected from a group consisting of
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of S protective antigen of the SARS-CoV-2 virus with gene C′-terminal deletion of 18 amino acids (SEQ ID NO:2);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of full-length S protective antigen of the SARS-CoV-2 virus and the human IgG1 Fc-fragment sequence (SEQ ID NO:3);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus S protein receptor-binding domain sequence with the viral leader peptide sequence (SEQ ID NO:4);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus protein S receptor-binding domain sequence with the transmembrane domain of vesicular stomatitis virus glycoprotein (SEQ ID NO:5);
an immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus S protein receptor-binding domain sequence with the leader peptide sequence and the human IgG1 Fc-fragment sequence (SEQ ID NO:6); and
an immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus full-length S protective antigen sequence on the basis of sequences of S protein genes of the SARS-CoV-2 virus (SEQ ID NO:1),
are administered to mammals with a time interval of more than one week.

9. Method presented herein in claim 7, wherein any one or more of the immunobiological agents based on recombinant human adenovirus serotype 5 and any one or more of the immunobiological agents based on recombinant human adenovirus serotype 26 selected from a group consisting of
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of S protective antigen of the SARS-CoV-2 virus with gene C′-terminal deletion of 18 amino acids (SEQ ID NO:2);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of full-length S protective antigen of the SARS-CoV-2 virus and the human IgG1 Fc-fragment sequence (SEQ ID NO:3);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus S protein receptor-binding domain sequence with the viral leader peptide sequence (SEQ ID NO:4);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus protein S receptor-binding domain sequence with the transmembrane domain of vesicular stomatitis virus glycoprotein (SEQ ID NO:5);
an immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus S protein receptor-binding domain sequence with the leader peptide sequence and the human IgG1 Fc-fragment sequence (SEQ ID NO:6); and
an immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus full-length S protective antigen sequence on the basis of sequences of S protein genes of the SARS-CoV-2 virus (SEQ ID NO:1),
are sequentially administered to mammals with a time interval of more than one week, or
any one or more of the immunobiological agents based on recombinant human adenovirus serotype 26 and any one or more of the immunobiological agents based on recombinant human adenovirus serotype 5 selected from a group consisting of
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of S protective antigen of the SARS-CoV-2 virus with gene C′-terminal deletion of 18 amino acids (SEQ ID NO:2);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of full-length S protective antigen of the SARS-CoV-2 virus and the human IgG1 Fc-fragment sequence (SEQ ID NO:3);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus S protein receptor-binding domain sequence with the viral leader peptide sequence (SEQ ID NO:4);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus protein S receptor-binding domain sequence with the transmembrane domain of vesicular stomatitis virus glycoprotein (SEQ ID NO:5);
an immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus S protein receptor-binding domain sequence with the leader peptide sequence and the human IgG1 Fc-fragment sequence (SEQ ID NO:6); and
an immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus full-length S protective antigen sequence on the basis of sequences of S protein genes of the SARS-CoV-2 virus (SEQ ID NO:1),
are sequentially administered to mammals with a time interval of more than one week.

10. Method presented herein in claim 7, wherein any two immunobiological agents based on recombinant human adenovirus serotype 5 or serotype 26 selected from a group consisting of
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of S protective antigen of the SARS-CoV-2 virus with gene C′-terminal deletion of 18 amino acids (SEQ ID NO:2);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the sequence of full-length S protective antigen of the SARS-CoV-2 virus and the human IgG1 Fc-fragment sequence (SEQ ID NO:3);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus S protein receptor-binding domain sequence with the viral leader peptide sequence (SEQ ID NO:4);
an immunobiological agent for the prevention of diseases caused by severe acute respiratory syndrome virus SARS-CoV-2 based on recombinant human adenovirus serotype 5 or recombinant human adenovirus serotype 26, containing optimized for the expression in mammalian cells the SARS-CoV-2 virus protein S receptor-binding domain sequence with the transmembrane domain of vesicular stomatitis virus glycoprotein (SEQ ID NO:5);
an immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus S protein receptor-binding domain sequence with the leader peptide sequence and the human IgG1 Fc-fragment sequence (SEQ ID NO:6); and
an immunobiological agent for the prevention of diseases caused by the severe acute respiratory syndrome (SARS-CoV-2) virus based on recombinant human adenovirus serotype 5, or recombinant human adenovirus serotype 26, containing optimized for the expression in mammalian cells the SARS-CoV-2 virus full-length S protective antigen sequence on the basis of sequences of S protein genes of the SARS-CoV-2 virus (SEQ ID NO:1),
are simultaneously administered to mammals.


SEQUENCE LISTING (6 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1255 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 3 - protein, 1445 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPTMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 236 aa, SARS-CoV-2
MFVFLVLLPLVSSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 5 - protein, 291 aa, SARS-CoV-2
MFVFLVLLPLVSSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFKLSSWKSSIASFFFIIGLIIGLFLVLRVGIHLCIKLKHTKKRQIYTDIEMNRLGK

SEQ ID NO: 6 - protein, 468 aa, SARS-CoV-2
MFVFLVLLPLVSSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFTMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK